(19) United States
(12) Patent Application Publication
(10) Pub. No.: US 2021/0147813 A1
(43) Pub. Date: May 20, 2021

(54) DRUG SCREENING PLATFORM USING BIOMATERIAL SCAFFOLDS

(71) Applicants: UNIVERSITY OF CENTRAL FLORIDA RESEARCH FOUNDATION, INC., Orlando, FL (US); MAYO CLINIC, Rochester, MN (US)

(72) Inventors: Stephen J. FLORCZYK, Orlando, FL (US); Kailei XU, Orlando, FL (US); Zi WANG, Orlando, FL (US); Ratna CHAKRABARTI, Orlando, FL (US); John COPLAND, Ponte Vedra Beach, FL (US)

(21) Appl. No.: 17/045,308

(22) PCT Filed: Apr. 3, 2019
(86) PCT No.: PCT/US2019/025545

§ 371 Date: Oct. 5, 2020

Related U.S. Application Data
(60) Provisional application No. 62/652,309, filed on Apr. 3, 2018.
(60) Provisional application No. 62/726,499, filed on Sep. 4, 2018.

(51) Int. Cl. C12N 5/09; C12N 5/071

(52) U.S. Cl. CPC C12N 5/0693; C12N 2500/34; C12N 2501/905; C12N 5/0671

(57) ABSTRACT

Disclosed are cell culture scaffolds comprising a scaffold composition comprising chitosan and alginate, hyaluronic acid, or chondroitin sulfate, wherein the scaffold is formed from an aqueous solution comprising greater than 4 wt % chitosan. These scaffolds can be contacted with cells, e.g., patient-derived cancer cells. The scaffolds provide 3D structure allowing appropriate cell function to occur. A scaffold of chitosan and chondroitin sulfate are also disclosed. Scaffold compositions comprising chitosan and alginate, hyaluronic acid, or chondroitin sulfate are also disclosed. Methods of making these scaffolds, e.g., by freeze casting or 3D printing, and using them to evaluate a patient's cancer cell and personalize treatment are also disclosed.

### CROSS REFERENCE TO RELATED APPLICATIONS

This application is a national stage application filed under 35 U.S.C. § 371 of PCT/US2019/025545, filed Apr. 3, 2019, which claims the benefit of priority to U.S. Provisional Application 62/652,309, filed Apr. 3, 2018, and U.S. Provisional Application 62/726,499, filed Sep. 4, 2018, the disclosures of each are incorporated by reference herein in their entireties.

### BACKGROUND

Only about five percent of new cancer drugs are approved, and most fail due to lack of efficacy. A reason is that current preclinical methods are limited in predicting successful outcomes. For example, biological heterogeneity and lack of distinguishable histologic subtypes complicates the development of new cancer therapeutics. With the age of next-generation sequencing and integrated genomics evidence for molecularly defined subtypes are emerging with tumors being classified by their genome copy number, fusion gene profiles, mutational landscapes, and mRNA splicing patterns (Rubin M A, et al., (2011) Chinnaiyan A M. Common Gene Rearrangements in Prostate Cancer. J Clin Oncol 29(27):3659-3668; Grasso C S, et al., (2012) The mutational landscape of lethal castration-resistant prostate cancer. Nature 487(7406):239-243; Taylor B S, et al., (2010) Integrative Genomic Profiling of Human Prostate Cancer. Cancer Cell 18(1):11-22).

To utilize these new discoveries, development of new preclinical models that capture the diversity of cancers are needed. Recently, 3D cultures have been demonstrated to better mimic the in vivo tumor microenvironment than 2D cultures (Fischbach C, et al., (2007) Engineering tumors with 3D scaffolds, Nat Methods 4(10):855-60; Florczyk S J, et al., (2013) Porous chitosan-hyaluronic acid scaffolds as a mimic of glioblastoma microenvironment ECM, Biomaterials 34(38):10143-50). This has led to investigation and development of 3D scaffolds for 3D culture of cancer cells. Composition and stiffness of the 3D substrate have been shown to influence cell morphology, cytoskeletal structure, signaling, and function (Baker B M, et al., (2012) Deconstructing the third dimension: how 3D culture microenvironments alter cellular cues, J Cell Sci 125(Pt 13):3015-24). Chitosan is a commonly used biomaterial which has a similar chemical structure to glycosaminoglycans and has beneficial biological properties including biocompatibility, biodegradability, and hydrophilic surface (Muzzarelli R A A, (2011) Chitosan composites with inorganics, morphogenetic proteins and stem cells, for bone regeneration, Carbohydrate Polymers 83(4):1433-1445). The cationic nature of chitosan allows it to form an ionic complex by mixing with anionic polymers. Previously, 4 wt % chitosan-alginate (CA) scaffolds were shown to support growth of PCa cells and support the interaction of immune cells with tumor cells (Florczyk S J, et al., (2012) 3D porous chitosan-alginate scaffolds: a new matrix for studying prostate cancer cell-lymphocyte interactions in vitro, Adv Healthc Mater 1(5):590-9). While these scaffolds show promise, new compositions for 3D scaffolds that can support the growth of various cancer cells and that have improved properties are still needed. Methods of using such new compositions to improve and personalize cancer treatments are also needed. The compositions and methods disclosed herein address these and other needs.

### SUMMARY

In accordance with the purposes of the disclosed materials and methods, as embodied and broadly described herein, the disclosed subject matter, in one aspect, relates to compounds, compositions and methods of making and using compounds and compositions. In specific aspects, the disclosed subject matter relates to cancer therapy and to methods of improving and personalizing cancer therapy. In further examples, disclosed are cell culture scaffolds comprising a scaffold composition comprising chitosan and alginate, hyaluronic acid, or chondroitin sulfate, wherein the scaffold comprises greater than 4 wt % of the scaffold composition; and patient-derived cancer cells. A scaffold of chitosan and chondroitin sulfate are also disclosed. Scaffold compositions comprising chitosan and alginate, hyaluronic acid, or chondroitin sulfate are also disclosed. Methods of making these scaffolds, e.g., by freeze casting or 3D printing, and using them to evaluate a patient's cancer cell and personalize treatment are also disclosed.

Additional advantages will be set forth in part in the description that follows or may be learned by practice of the aspects described below. The advantages described below will be realized and attained by elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying figures, which are incorporated in and constitute a part of this specification, illustrate several aspects described below.

FIG. 1 is a group of SEM images of pore structure for 2 wt %, 4 wt %, and 6 wt % CA scaffolds. The images show the interconnected pores. Pore size decreases with increasing scaffold concentration. Scale bar for top images is 250 μm and 50 μm for bottom images.

FIG. 2 is a group of SEM images of PC-3 cells cultured on 2 wt %, 4 wt %, and 6 wt % CA scaffolds. The PC-3 cells do not show significant differences in morphology with different scaffold stiffness.

FIGS. 3A-3D are graphs show the compressive mechanical properties of CA scaffolds. Stiffness of 2, 4, and 6 wt % CA scaffolds are shown for dry (FIG. 3A) and wet (FIG. 3B) conditions. PDX tumor stiffness was included in the wet plot. Compressive strength of 2, 4, and 6 wt % CA scaffolds are shown for dry (FIG. 3C) and wet (FIG. 3D) conditions.

FIG. 4 is a group of SEM images of C4-2B cells cultured on 2 wt %, 4 wt %, and 6 wt % CA scaffolds. The C4-2B cells show significant differences in mineralization with different scaffold stiffness. Mineralization increases with scaffold stiffness.

FIG. 5A is a schematic illustration of the The Freeze-FRESH (FF) technique producing 3D printed biomaterial scaffolds with hierarchical porosity. FIG. 5B is a group of images showing a comparison of the honeycomb scaffold design and the appearance of 3D printed scaffolds after recovery from the support bath. The figures contrast scaffolds prepared by different processing routes: smooth samples were recovered after 3D printing (no freezing), while the porous samples were prepared by FF technique (print and freeze). The recovered printed scaffolds were immersed in cell culture media and the presence of bubbles inside the struts of the porous scaffold denote pore formation within the scaffold struts. Scale bars are 10 mm.

FIG. 6 is a group of spectra for chitosan and alginate. Individual materials are at the top. The polyelectrolyte complex formation is demonstrated by peaks at 1580 cm⁻¹ and 1650 cm⁻¹.

FIG. 7 shows 22Rv1 cell morphology on CA scaffolds. SEM images of PC-3 cells on 2, 4, and 6 wt % CA scaffolds at 5, 10, and 15 d time points.

FIG. 8 is a group of spectra for chitosan, chondroitin sulfate, and various mixtures of the two. Individual materials are at the top. The polyelectrolyte complex formation is demonstrated by peaks at 1580 cm⁻¹ and 1650 cm⁻¹.

FIG. 9 is a pair of graphs showing the scaled diameter over time for various C-CS scaffolds under dry and wet conditions. The swelling test demonstrates the scaffold stability for in vitro conditions (37° C. and 5% CO₂) up to 500 hours.

FIG. 10 is a pair of graphs showing the stiffness (Young's Modulus) of various C-CS scaffolds with different CS content. Scaffolds stiffness decreases with increasing of CS content for dry scaffolds. Similar stiffness was observed in wet conditions, with decreased stiffness for 4-1 scaffolds.

FIG. 11 is a group of SEM images of different C-CS scaffolds. The images demonstrate interconnected pores for all compositions. Pore size is relatively consistent between compositions. Scale bar for top images is 250 μm and 50 μm for bottom images.

FIG. 12 is a group of immunofluorescence images of functional proteins of PC-3 cells cultured on 4-0.25 and 4-1 C-CS scaffolds at day 10. Ki-67 (green top images) cytokeratin 8 (green bottom images), phosphor-epidermal growth factor receptor (red, both).

FIG. 13 is a group of immunofluorescence images of functional proteins from tissues and cells cultured for 10 days. Androgen receptor (red, prostate+C4-2B), phosphor-epidermal growth factor receptor (red, PC-3), cytokeratin 9 (green, both cell lines).

FIG. 14 is a graph of gene expression of functional PCa markers. LIMK1 expression for PC-3 in C-CS scaffolds are higher than in 2D and 4-0.25 has higher expression than 4-1. For C4-2B, 4-1 C-CS scaffold has higher PSA expression than 2D. However, 4-0.25 C-CS has lower expression than 2D.

FIG. 15 is a group of Brightfield and SEM images showing the morphological difference of printed alginate chitosan, and controlled PCL samples. The directly printed samples had a smooth surface while the porous structure formed in printed scaffolds processed with freezing and lyophilization. The pore morphology change is related to different freezing temperature. The freeze-casted samples showed porous features but without any geometrical design. The PCL scaffold had smooth surface with crosshatch design to acquire macro-porosity in the structure. Scale bars represent 500 μm.

FIG. 16 is a graph showing the stiffness of alginate. Samples were tested under wet condition to mimic the state while they were cultured with cells in media.

FIG. 17 is a graph showing cell proliferation on day 3 and 7 of 3DP samples and 2D control groups on alginate.

FIG. 18A is a group of GFP images showing cell morphology on a 2DP and freeze casted alginate scaffolds. FIG. 18B is a group of GFP images showing cell morphology on PCL and 2D. Scale bars are 200 μm.

FIG. 19 is a photograph showing representative C-HA scaffold (4-1-1) appearance in wet and dry conditions.

FIG. 20 is a group of micrographs showing C-HA scaffold pore structure. SEM images of the seven C-HA compositions, scale bars are 100 μm.

FIG. 21 is a graph showing C-HA scaffold percent porosity and pore size.

FIG. 22 is a graph showing FTIR analysis of C-HA scaffolds. The C-HA spectra are shown along with chitosan and HA to characterize polyelectrolyte complex formation. The right panel highlights a region of the spectra with peak broadening in C-HA samples.

FIG. 23 is a graph showing C-HA solution viscosity.

FIG. 24 is a pair of graphs showing C-HA scaffold stiffness in dry and wet conditions.

FIG. 25 is a graph showing MDA-MB-231 cell numbers increase on all C-HA scaffold compositions over 15 days.

FIG. 26 is a group of images showing MDA-MB-231 cell morphology on 4-1-5 C-HA scaffolds over 15 days, scale bars are 500 μm and 200 μm, top and bottom, respectively.

FIG. 27 is a group of micrographs showing MDA-MB-231 cell morphology on 4-1-5 C-HA scaffold over 15 days, scale bars are 25 μm.

FIGS. 28A-28C show that FF printing is a versatile technique that can be used for printing custom designs. Here, a human femur model print is shown. FIG. 28A shows a smooth print that was recovered directly from support bath after printing. The smooth print has a solid structure with some surface texture. FIG. 28B shows a porous print produced with the FF method and recovered from the support bath. Pores formed within the printed structure are visible by the presence of entrapped air bubbles (arrows). FIG. 28C shows that incubating the FF print in dye solution caused the dye to diffuse through the pores throughout the print, which further demonstrated the formation of interconnected pores inside the printed structure.

FIG. 29 shows that the FF method creates porosity throughout the printed structure. The FF method femur print is presented to examine the surface morphology and the pore formation within the printed structure. The surface of the print was rough and flat surfaces in some locations are closed pores. The cross-section images showed a porous structure, confirming the pore formation throughout the print.

FIGS. 30A-30D show that the FF method increases scaffold resiliency. Bending of smooth (FIGS. 30A and 30B) and FF scaffolds (FIGS. 30C and 30D) demonstrates that the FF scaffold had greater bending angle than smooth scaffold. The bending of the scaffolds demonstrates that the FF method provides greater resilience to the printed scaffolds, in addition to pores within the printed structure.

FIG. 31 shows that the FF method produces scaffolds with hierarchical pore structure. Morphological differences in the pore structure of alginate scaffolds: smooth, FF (porous), freeze cast (FC), and commercial poly ε-caprolactone (PCL) 3D printed scaffold. The smooth scaffolds had a relatively smooth surface in the dehydrated state, while the FF scaffolds demonstrated porosity within the printed scaffold structures. The pores within the scaffold structure had similar pore sizes at different freezing temperatures. The FC and PCL scaffolds provided controls: FC scaffolds had porous features without controlled deposition, while the PCL scaffolds had a macroporous crosshatch structure with smooth strut surfaces.

FIGS. 32A-32B shows the mechanical properties of alginate FF scaffolds. Bulk and wall stiffness of smooth and FF (porous) scaffolds, were compared with freeze cast (FC) scaffolds. Samples were tested in wet conditions to mimic the properties during cell culture.

FIG. 33 shows that FF scaffolds support cell growth. Alginate scaffolds were prepared with and without RGD peptide conjugation as smooth, FF, and freeze cast (FC) scaffolds. The scaffolds were evaluated with MDA-MB-231 culture and cell numbers were assayed at 3 d and 7 d. All scaffold samples with RGD conjugation showed increased cell number compared to scaffolds made from pure alginate. (*) denotes significant difference where p<0.05.

FIG. 34 shows MDA-MB-231 cell morphology on FF scaffolds. Fluorescence images showing MDA-MB-231-GFP cell morphology on smooth, FF, FC, and PCL scaffolds at 3 d. Cells seeded on TCPS (2D) were used for comparison. Cells only adhered to the surface of struts of smooth print, even with the presence of RGD. Cells formed clusters on the struts of FF scaffolds; the pores on the scaffold struts provide additional sites for cell adhesion. The cells on PCL scaffolds adhered to the smooth surface and exhibited similar elongated morphology as in 2D cultures.

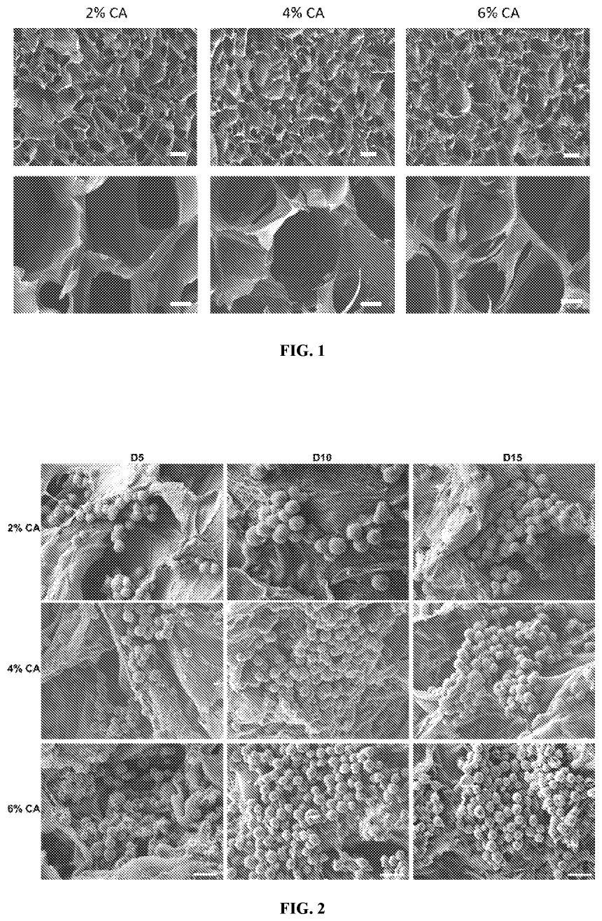

### DETAILED DESCRIPTION

The materials, compounds, compositions, and methods described herein may be understood more readily by reference to the following detailed description of specific aspects of the disclosed subject matter and the Examples and Figures included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

In this specification and in the claims that follow, reference will be made to a number of terms, which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the cell” includes mixtures of two or more such cells, and the like.

“Optional” or “optionally” means that the subsequently described event or circumstance can or cannot occur, and that the description includes instances where the event or circumstance occurs and instances where it does not.

By “reduce” or other forms of the word, such as “reducing” or “reduction,” is meant lowering of an event or characteristic (e.g., tumor growth, metastasis). It is understood that this is typically in relation to some standard or expected value, in other words it is relative, but that it is not always necessary for the standard or relative value to be referred to. For example, “reduces tumor growth” means decreasing the amount of tumor cells relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

As used herein, “treatment” refers to obtaining beneficial or desired clinical results. Beneficial or desired clinical results include, but are not limited to, any one or more of: alleviation of one or more symptoms (such as tumor growth or metastasis), diminishment of extent of cancer, stabilized (i.e., not worsening) state of cancer, delaying spread (e.g., metastasis) of the cancer, delaying occurrence or recurrence of cancer, delay or slowing of cancer progression, amelioration of the cancer state, and remission (whether partial or total).

The term “patient” preferably refers to a human in need of treatment with an anti-cancer agent or treatment for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others, that are in need of treatment with an anti-cancer agent or treatment.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

As used herein, the term “composition” is intended to encompass a product comprising the specified ingredients in the specified amounts, as well as any product which results, directly or indirectly, from combination of the specified ingredients in the specified amounts.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5, and are present in such ratio regardless of whether additional components are contained in the mixture.

A weight percent (wt. %) of a component, unless specifically stated to the contrary, is based on the total weight of the formulation or composition in which the component is included.

As used herein, a 3D culture is a cell or tissue culture that is on a structure that has height, width, and length of similar dimensions. These structures have a calculable volume and are to be contrasted to 2D cultures, which are generally flat and on flat surfaces.

### Compositions

A need exists to provide personalized therapy for cancer patients, especially those diagnosed with metastatic disease or those who have progressed on all standard of care therapies and are facing death. Needs also exist for methods of personalizing therapy for a wide variety of cancers. The compositions and methods disclosed herein can be used in preclinical models derived from patient tumor tissues allowing medium throughput drug screening of a patient's tumor cells in three-dimensional (3D) culture. The disclosed 3D models can be used to demonstrate recapitulation of the patient's tumor microenvironment (TME).

In certain aspects, disclosed herein are scaffold compositions comprising chitosan alginate. These scaffold compositions can be cast or 3D printed in various scaffold structures, as described herein. The resulting scaffolds can then be placed into a culture dish and sterilized. Within a sterile cell culture dish where the scaffold has been placed, a specific number of a patient's cancer cells in culture media can be added onto the scaffold surface. Culture dishes can be placed into a 37° C. cell culture incubator allowing cells to attach and proliferate on the scaffold. These 3D cultures can then be contacted with one or more therapeutics and then analyzed to evaluate different treatments for the patient with the expectation that effective treatments will significantly reduce cell numbers compared to control untreated cells.

### Chitosan

The disclosed scaffold compositions comprise as a primary component chitosan. Chitosan is a naturally occurring polymer found in many fungi. However, as a matter of convenience, chitosan is obtained from chitin, which (after cellulose) is the second most abundant natural polymer. Chitin is readily isolated from shellfish or insect exoskeletons, and is also found in mollusks and fungi. Chitin is a water-insoluble copolymer of N-acetyl-D-glucosamine and D-glucosamine, but the great preponderance of monomer units are N-acetyl-D-glucosamine residues. Chitosan is a copolymer of the same two monomer units, but the preponderance of monomer units are D-glucosamine residues. Since the D-glucosamine residues bear a basic amino function, they readily form salts with acids. Many of these salts are water soluble. Treatment of chitin with concentrated caustic at elevated temperature converts N-acetyl-D-glucosamine residues into D-glucosamine residues and thereby converts chitin into chitosan. There is a continuum of compositions possible between pure poly-N-acetyl-D-glucosamine and pure poly-D-glucosamine. These compositions are all within the skill of the art to prepare and are all suitable for the uses described herein.

The chitosan component can also be used in its salt form by contacting chitosan with an acid. Suitable acids for making chitosan salts are those acids that form water-soluble salts with chitosan. It is not necessary that the acid itself be water-soluble; however, such water-soluble acids can ease handling. Inorganic acids, which form water-soluble chitosan salts, include the halogen acids and nitric acid, but exclude sulfuric and phosphoric acids because they do not form water-soluble salts with chitosan. Organic acids are particularly suitable and include, but are not limited to, lactic acid, glycolic acid, glutamic acid, formic acid, acetic acid, and a mixture thereof. Either mono-or poly-functional carboxylic acids can also be used. They can be aliphatic or aromatic, so long as they form water-soluble salts with chitosan.

In the disclosed scaffold compositions, mixtures of chitosans can also be used. Further, it is generally desired that the chitosan component be cationic. In addition to chitosan component, the disclosed scaffold compositions can comprise an anionic component chosen from alginate, hyaluronic acid, and/or chondroitin. The cationic chitosan component and the anionic component together form an electrostatic interaction called a polyelectrolyte complex. The polyelectrolyte complex can be concentrate and dried, placed into culture dishes and sterilized for use as a cell scaffold.

In a dried scaffold composition, the composition is completely (or nearly completely) made up of the polyelectrolyte composition (i.e., chitosan and either alginate, hyaluronic acid, or chondroitin sulfate). Prior to drying the scaffold composition, the components can be present in an aqueous solution. The aqueous solution can comprise water, chitosan, and either alginate, hyaluronic acid, or chondroitin sulfate. It has been found that varying the amount of the chitosan and/or anionic component in the aqueous solution can affect the physical properties of the resulting scaffold composition after it is dried.

Chitosan can be present in the aqueous scaffold compositions at an amount of greater than 4 wt %, e.g., greater than 5 wt %, greater than 6 wt %, greater than 7 wt %, greater than 8 wt %, greater than 9 wt %, or greater than 10 wt. %. In other examples, the chitosan can be present in the disclosed scaffold compositions at an amount of from greater than 4 wt. % to 10 wt %, from greater than 4 wt % to 9 wt %, from greater than 4 wt % to 8 wt %, from greater than 4 wt % to 7 wt %, from greater than 4 wt % to 6 wt %, from 5 wt % to 10 wt %, from 5 wt % to 9 wt %, from 5 wt % to 8 wt %, from 5 wt % to 7 wt %, from 6 wt % to 10 wt %, from 6 wt % to 9 wt %, or from 6 wt % to 8 wt %.

### Alginate

Alginate can be combined with chitosan in a variety of suitable methods to form chitosan-alginate scaffold composition. Alginic acid or alginate is a polysaccharide that can be obtained from brown algae. Alginate are available in filamentous, granular, or powder forms. The alginates that are suitable for use herein can be salts, e.g., sodium alginate, potassium alginate, or calcium alginate. In some embodiments, alginate is combined with the solid form of chitosan to form a combined solid mixture. The combined solid mixture can then be dissolved in a suitable solvent to form a chitosan-alginate solution. In further embodiments, alginate is introduced into an already formed chitosan solution to dissolve the alginate and form the chitosan-alginate solution. In further embodiments, the chitosan is introduced to an alginate solution that contains suitable solvent to form the chitosan-alginate solution. In further embodiments, a solution of alginate is combined with a solution of the chitosan to form the chitosan-alginate solution. The resulting chitosan-alginate solutions can then be dried (e.g., freeze dried) to result in the disclosed scaffold composition, which can then be placed into a culture dish and sterilized.

Alginate can be present in the disclosed aqueous scaffold compositions at an amount of greater than 2 wt %, greater than 3 wt %, greater than 4 wt %, e.g., greater than 5 wt %, greater than 6 wt %, greater than 7 wt %, greater than 8 wt %, greater than 9 wt %, or greater than 10 wt. %. In other examples, the alginate can be present in the disclosed scaffold compositions at an amount of from greater than 2 wt % to 10 wt %, from greater than 2 wt %, to 8 wt %, from greater than 2 wt % to 6 wt %, from 4 wt. % to 10 wt %, from 4 wt % to 8 wt %, from 4 wt % to 6 wt %, from 5 wt % to 10 wt %, from 5 wt % to 8 wt %, from 5 wt % to 7 wt %, from 6 wt % to 10 wt %, or from 6 wt % to 8 wt %.

### Hyaluronic Acid

Hyaluronic acid can be combined with chitosan in a variety of suitable methods to form a chitosan-hyaluronic acid scaffold composition. Hyaluronic acid or hyaluronan is an anionic nonsulfated glycosaminoglycan. In some embodiments, hyaluronic acid is combined with the solid form of chitosan to form a combined solid mixture. The combined solid mixture can then be dissolved in a suitable solvent to form a chitosan-hyaluronic acid solution. In further embodiments, hyaluronic acid is introduced into an already formed chitosan solution to dissolve the hyaluronic acid and form the chitosan-hyaluronic acid solution. In further embodiments, chitosan is introduced to a hyaluronic acid solution that contains suitable solvent to form the chitosan-hyaluronic acid solution. In further embodiments, a solution of hyaluronic acid is combined with a solution of chitosan to form the chitosan-hyaluronic acid solution. The resulting chitosan-hyaluronic acid solutions can then be dried (e.g., freeze dried) to result in the disclosed scaffold composition, which can then be placed into a culture dish and sterilized.

Hyaluronic acid can be present in the disclosed aqueous scaffold compositions at an amount of greater than 2 wt %, greater than 3 wt %, greater than 4 wt %, e.g., greater than 5 wt %, greater than 6 wt %, greater than 7 wt %, greater than 8 wt %, greater than 9 wt %, or greater than 10 wt. %. In other examples, the hyaluronic acid can be present in the disclosed aqueous scaffold compositions at an amount of from greater than 2 wt % to 10 wt %, from greater than 2 wt %, to 8 wt %, from greater than 2 wt % to 6 wt %, from 4 wt. % to 10 wt %, from 4 wt % to 8 wt %, from 4 wt % to 6 wt %, from 5 wt % to 10 wt %, from 5 wt % to 8 wt %, from 5 wt % to 7 wt %, from 6 wt % to 10 wt %, or from 6 wt % to 8 wt %.

### Chondroitin Sulfate

Chondroitin sulfate can be combined with chitosan in a variety of suitable methods to form a chitosan-chondroitin sulfate scaffold composition. Chondroitin sulfate is a sulfated glycosaminoglycan composed of alternating N-acetylgalactosamine and glucuronic acid. In some embodiments, chondroitin sulfate is combined with the solid form of chitosan to form a combined solid mixture. The combined solid mixture can then be dissolved in a suitable solvent to form a chitosan-chondroitin sulfate solution. In further embodiments, chondroitin sulfate is introduced into an already formed chitosan solution to dissolve the chondroitin sulfate and form the chitosan-chondroitin sulfate solution. In further embodiments, chitosan is introduced to a chondroitin sulfate solution that contains suitable solvent to form the chitosan-chondroitin sulfate solution. The resulting chitosan-chondroitin sulfate solutions can then be dried (e.g., freeze dried) to result in the disclosed scaffold composition, which can then be placed into a culture dish and sterilized.

Chondroitin sulfate can be present in the disclosed aqueous scaffold compositions at an amount of greater than 2 wt %, greater than 3 wt %, greater than 4 wt %, e.g., greater than 5 wt %, greater than 6 wt %, greater than 7 wt %, greater than 8 wt %, greater than 9 wt %, or greater than 10 wt. %. In other examples, the chondroitin sulfate can be present in the disclosed aqueous scaffold compositions at an amount of from greater than 2 wt % to 10 wt %, from greater than 2 wt %, to 8 wt %, from greater than 2 wt % to 6 wt %, from 4 wt. % to 10 wt %, from 4 wt % to 8 wt %, from 4 wt % to 6 wt %, from 5 wt % to 10 wt %, from 5 wt % to 8 wt %, from 5 wt % to 7 wt %, from 6 wt % to 10 wt %, or from 6 wt % to 8 wt %.

### Scaffolds

In specific examples, the disclosed scaffold compositions can comprise chitosan and alginate. In other specific examples, the disclosed scaffold compositions can comprise chitosan and hyaluronic acid. In still other specific examples, the disclosed scaffold compositions can comprise chitosan and chondroitin sulfate. As noted herein, these compositions can be aqueous or can be dried.

Within a sterile cell culture dish where a dried scaffold has been placed, a specific number of the patient's cancer cells in culture media can be added onto the scaffold surface. Culture dishes can be placed into a 37° C. cell culture incubator allowing cells to attach and proliferate on the scaffold. These 3D cultures can then be contacted with one or more therapeutics and then analyzed to evaluate different treatments for the patient with the expectation that effective treatments will significantly reduce cell numbers compared to control untreated cells.

The chitosan-alginate (CA), chitosan-hyaluronic acid (C-HA), and chitosan-chondroitin sulfate (C-CS) scaffolds can be used as a platform for 3D culture and to evaluate the influence of different mechanical properties (stiffness) and scaffold compositions on various cancer cell lines derived from patient tumor tissues. The scaffolds can contain CA, C-HA, or C-CS alone, or with further additives (e.g., preservatives). The dried scaffold compositions can be prepared from aqueous solutions of CA, C-HA, or C-CS at different concentrations. For example, the aqueous solutions can contain an amount of greater than 2 wt %, greater than 3 wt %, greater than 4 wt %, e.g., greater than 5 wt %, greater than 6 wt %, greater than 7 wt %, greater than 8 wt %, greater than 9 wt %, or greater than 10 wt. % of chitosan, alginate, hyaluronic acid, and/or chondroitin sulfate. In other examples, the aqueous scaffold compositions can contain each of chitosan, alginate, hyaluronic acid, and chondroitin sulfate at an amount of from greater than 2 wt % to 10 wt %, from greater than 2 wt %, to 8 wt %, from greater than 2 wt % to 6 wt %, from 4 wt. % to 10 wt %, from 4 wt % to 8 wt %, from 4 wt % to 6 wt %, from 5 wt % to 10 wt %, from 5 wt % to 8 wt %, from 5 wt % to 7 wt %, from 6 wt % to 10 wt %, or from 6 wt % to 8 wt %. In specific examples, when the scaffold is CA, the amount of chitosan and/or alginate can be greater than 4 wt %, e.g., from 5 wt % to 10 wt %. The aqueous solutions can also comprise from, e.g., 1% to 10% acetic acid. In general, the greater the amount of chitosan and/or the anionic component present in the aqueous solution, the stiffer the resulting scaffold is after drying. Further, while not wishing to be bound by theory, it is believed that cancer cultures will show greater malignancy in scaffolds with higher stiffness.

The disclosed dried scaffolds can thus have a stiffness (Young's Modulus) of greater than 2 MPa, e.g., greater than 3 MPa or greater than 4 MPa (with an upper limit in some cases of 8 MPa).

The ratio of chitosan to either alginate, hyaluronic acid, or chondroitin sulfate in either the dried scaffold composition or in the aqueous solution used to prepare it can be from 5:1 to 1:5, e.g., 5:1, 5:2, 5:3, 5:4, 4:1, 4:3, 3:1, 3:2, 2:1, 1:1, 1:2, 2:3, 1:3, 3:4, 1:4, 4:5, 3:5, 2:5, 1:5. In specific examples, the ratio of C to A can be 1:1, 2:1, or 4:1. The ratio of C to HA can be from 5:1 to 1:5, e.g., 5:1, 5:2, 5:3, 5:4, 4:1, 4:3, 3:1, 3:2, 2:1, 1:1, 1:2, 2:3, 1:3, 3:4, 1:4, 4:5, 3:5, 2:5, 1:5. In specific examples, the ratio of C to HA can be 1, 2:1, or 4:1. The ratio of C to CS can be from 5:1 to 1:5, e.g., 5:1, 5:2, 5:3, 5:4, 4:1, 4:3, 3:1, 3:2, 2:1, 1:1, 1:2, 2:3, 1:3, 3:4, 1:4, 4:5, 3:5, 2:5, 1:5. In specific examples, the ratio of C to CS can be 1:1, 2:1, or 4:1.

The scaffolds can also have pores to support or contain cells. The pore diameter can be from 50 μm to 300 μm in diameter, e.g., from 50 μm to 250 μm, from 50 μm to 200 μm, from 50 μm to 150 μm, from 50 μm to 100 μm, from 100 μm to 300 μm, from 100 μm to 250 μm, from 100 μm to 200 μm, from 100 μm to 150 μm, from 150 μm to 300 μm, from 150 μm to 250 μm, from 150 μm to 200 μm, from 200 μm to 300 μm, from 200 μm to 250 μm, or from 250 μm to 300 μm in diameter. The pore diameter can be an average pore diameter. In particular examples, the scaffolds can have an average pore size of from 200 μm to 250 μm in diameter. Further, the porosity can be at least 80%.

The disclosed scaffolds are 3D. There is a remarkable difference between cell culture in 2D (e.g., those in petri dishes or on cover slips) and 3D scaffolds. 2D growth can alter metabolic pathways, change cell morphologies and reduce ECM protein production compared to in vivo conditions.

The scaffold can be prepared by combining an aqueous solution of chitosan and an aqueous solution of alginate, hyaluronic acid, or chondroitin sulfate to provide a mixture. The mixture can be stirred. The water can also be removed by evaporation or lyophilization to thereby provide a scaffold composition. Within a sterile cell culture dish where the scaffold has been placed, a specific number of the patient's cancer cells in culture media will be added onto the scaffold surface. Culture dishes will be placed into a 37° C. cell culture incubator allowing cells to attach and proliferate on the scaffold. These 3D cultures can then be contacted with one or more therapeutics and then analyzed to evaluate different treatments for the patient with the expectation that effective treatments will significantly reduce cell numbers compared to control untreated cells. As described herein, the scaffold composition can be 3D printed or freeze-casted. Freeze-casting is performed by casting the scaffold composition in a mold and freeze at freezing temperature (typically −20° C.). The solvent (water) freezes and excludes the polymeric material. The frozen sample is then lyophilized (freeze dried) to yield a 3D porous scaffold.

In certain examples, the scaffolds can support cultures of prostate cancer (PCa) cell lines and primary PCa (PPCa) cells derived from patient tumor tissues. PPCa cells can be established in culture from patients who have been exposed to multiple therapies for treatment of metastatic PCa. Knowing the response to therapies and drug resistance in patients allows one to compare drug responses in the matching patient derived 3D cultured cells. In similar examples, the scaffolds can comprise a patient-derived cancer that is selected from bladder, bone, bone marrow, bowel (including colon and rectum), breast, eye, gall bladder, kidney, mouth, larynx, esophagus, stomach, testis, cervix, head, neck, ovary, lung, mesothelioma, neuroendocrine, penis, skin, spinal cord, thyroid, vagina, vulva, uterus, liver, muscle, pancreas, blood cells (including lymphocytes and other immune system cells), and brain cancer. In particular examples, the cancer cells are breast or glioblastoma cells. Established cell lines of these cancers can also be used in the disclosed scaffolds to create additional models. In specific examples, solid tumor cells can be used.

Patient derived cancer cells can be obtained directly from the patient (e.g., by biopsy or resection). Alternatively, the patient derived cancer cells can come from a patient derived xenograft (PDX) model. Frozen and paraffin embedded patient tumor tissue allows for further molecular and genomic comparisons between tumor tissues and tumor tissue derived living models.

The cultures can be characterized by proliferation, cell morphology, immunohistochemistry (IHC) of cell biomarkers, PCa biomarkers (PSA, androgen response), and microRNA (miRNA) expression using RNAseq. Cultures can also be screened with chemotherapies that are currently in clinical use to evaluate the drug response in 2D versus 3D cultures. Novel therapies can also be incorporated into drug screens based upon patient tumor tissue DNA mutation status and IHC analysis.

### Methods

The disclosed chitosan-based porous scaffolds can be utilized with different compositions and different mechanical properties to culture cancer cells, particularly those isolated from cancer patients. The scaffolds can promote phenotypic responses to the scaffold environment and provide an additional tool to assess the patient's cancer. The disclosed scaffold compositions can be used to create models with a patient's own cancer cells, thereby allowing the evaluation of cells collected from a cancer patient in different microenvironments to assess the phenotype and metastatic potential of the cells. The scaffolds and models also allow screening of chemotherapies to evaluate the best options for the patient, allowing a personalized medicine approach for cancer treatment.

In the disclosed methods, a patient's response to a cancer therapeutic can be evaluated. The method can involve providing a scaffold comprising chitosan and either alginate, hyaluronic acid, or chondroitin sulfate as disclosed herein. In some examples, the scaffold can be prepared from an aqueous solution of chitosan comprising greater than 4 wt %, e.g., 5 wt %, 6 wt %, or 8 wt. %. In some examples, the scaffold can be prepared from an aqueous solution of alginate comprising greater than 4 wt %, e.g., 5 wt %, 6 wt %, or 8 wt. %. In some examples, the scaffold can be prepared from an aqueous solution of hyaluronic acid comprising greater than 4 wt %, e.g., 5 wt %, 6 wt %, or 8 wt. %. In some examples, the scaffold can be prepared from an aqueous solution of chondroitin sulfate comprising greater than 4 wt %, e.g., 5 wt %, 6 wt %, or 8 wt. %. The aqueous solutions can be dried to provide the scaffold. The dried scaffold can also comprise a medium or be provided neat.

Cancer cells derived from the patient and culture medium can be contacted to the surface of the scaffold and then the cells can be cultured. The cultured cells can be contacted with a with a putative therapeutic. Should a particular therapeutic be identified as having a therapeutic effect on the cancer cell, that therapeutic can be administered to the patient.

Also disclosed are methods of characterizing a patient's cancer cells. The methods can involve providing a cell culture scaffold comprising chitosan and alginate, hyaluronic acid, or chondroitin sulfate. In some examples, the scaffold can be prepared from an aqueous solution of chitosan comprising greater than 4 wt %, e.g., 5 wt %, 6 wt %, or 8 wt. %. In some examples, the scaffold can be prepared from an aqueous solution of alginate comprising greater than 4 wt %, e.g., 5 wt %, 6 wt %, or 8 wt. %. In some examples, the scaffold can be prepared from an aqueous solution of hyaluronic acid comprising greater than 4 wt %, e.g., 5 wt %, 6 wt %, or 8 wt. %. In some examples, the scaffold can be prepared from an aqueous solution of chondroitin sulfate comprising greater than 4 wt %, e.g., 5 wt %, 6 wt %, or 8 wt. %. The aqueous solutions can be dried to provide the scaffold. The dried scaffold can also comprise a medium or be provided neat.

Cancer cells derived from the patient and culture medium can be contacted to the surface of the scaffold and then the cells can be cultured. The cancer cells can then be removed from the cell culture scaffold, and the phenotype or metastatic potential of the cancer cells can be characterized.

In certain examples, more than one scaffold composition with different stiffnesses can be provided. Cancer cells derived from the patient and culture medium can be contacted to the surface of the scaffolds. The cells can be characterized, e.g., metastatic potential, based on the amount of growth on the different scaffolds. For example, a higher rate of growth on stiffer scaffolds can indicate a higher metastatic potential.

### 3D Printing Methods

Disclosed herein are methods for printing a three-dimensional scaffold structure that involve printing a scaffold composition onto a printing surface by forcing the composition through at least one extrusion die and onto the printing surface, wherein the extrusion die and/or printing surface are moved in the X, Y, and/or Z direction while printing the scaffold. In the disclosed methods, the scaffold composition is deposited onto a substrate through an extrusion die. That is, the scaffold composition is extruded through a die (also called nozzles) by high pressure blowing gas. The gas can be ambient or heated gas, and can be air, CO₂, N₂, or other unreactive gas. In other examples, the scaffold composition is extruded through a die with a pump or extruder. In the disclosed methods, the location of the scaffold composition is controlled by an X-Y-Z movement system, as are used in 3D printing systems. Such X-Y-Z movement systems can move either the surface that the scaffold composition is being deposited on (printing surface), the extrusion die, or both, such that the scaffold composition deposits in a preselected 3D structure or pattern. The movement can be controlled by one or more computers communicatively connected to the X-Y-Z movement system.

The disclosed methods use a nozzle or die based system to form scaffold structures in a linear array or a circular array. In other examples, a secondary material may be introduced through another die. The die assembly can be fixed while the printing surface (scaffold collection surface) is moved.

In other aspects, the scaffold composition can be forced through more than one extrusion die. Extrusion dies of different shapes can deposit the scaffold composition as different patterns, orientations, or thicknesses. The extrusion dies can be circular, flat, singular capillary. The extrusion die can be a Reicofil die geometry (see U.S. Pat. Nos. 3,650,866 and 3,972,759, which are incorporated by reference herein in their entireties for their teachings of die geometries). The extrusion dies can also be a Biax geometry that uses multiple rows of (spinning) orifices with co-centric air supply (see U.S. Pat. No. 5,476,616, which is incorporated by reference herein in its entirety for its teachings of die geometries). Any combinations of these dies can be used. That is, multiple extrusion dies can be used so that different patterns of scaffold compositions can be applied to the printing surface, making multiple layers of different patterns. The use of multiple dies can also permit printing structures of variable thickness, with different properties at different locations, in a single processing step using one or combination of 3D-printing processes.

In specific aspects, the scaffold compositions can be extruded into a support material that comprises gelatin beads. The beads can have a diameter of 85 micrometers. The beads provide a Bingham plastic response (seals behind needle). After the scaffold composition is printed, the scaffold can be frozen to introduce additional porosity, then freeze dried (lyophilized) remove the ice. The scaffold is then recovered from the gelatin beads by crosslinking or neutralizing the scaffold composition and melting the gelatin.

The printing can also include the addition of one or more other natural or synthetic polymers. These additional polymers can be, but are not limited to, poly(glycolic acid) (PGA), poly(lactic acid) (PLA), poly(c-caprolactone) (PCL), polyurethanes, poly(ortho esters) (POE), poly(anhydrides), polyvinyl alcohol (PVA), tyrosinederived polycarbonates, copolymers thereof, and any combination thereof. In further specific examples, the additional polymers can be, but are not limited to, collagen, fibrin, agarose, starch, gelatin, cellulose, and any combination thereof. In other examples, the additional polymers can further comprise proteins. Any of these fibers can be used in the form of a melt or a solution.

In a specific example, disclosed is a method of preparing a 3D cell culture scaffold that comprises extruding a scaffold composition comprising chitosan and alginate, hyaluronic acid, or chondroitin sulfate, wherein the scaffold comprises greater than 4 wt % of the scaffold composition onto a substrate material, wherein the location of the scaffold composition extruded onto the substrate material is controlled by an X-Y-Z movement system, and wherein the substrate material comprises gelatin beads; freezing the scaffold composition and gelatin beads; removing ice from the frozen scaffold composition and gelatin beads; crosslinking or neutralizing the scaffold composition; and removing the gelatin from the crosslinked or neutralized scaffold composition, thereby providing a 3D cell culture scaffold.

### Apparatus

An apparatus for printing a three-dimensional scaffold structure that comprises an extrusion unit comprising at least one extrusion die; a printing platform; an X-Y-Z movement system configured to move the at least one extrusion die and/or the printing platform in a three coordinate system; and at least one computer communicatively coupled with the X-Y-Z movement system, the at least one computer programed to receive three-dimensional print type inputs for a structure to be three-dimensionally printed and to control the X-Y-Z movement system and extrusion unit.

The extrusion unit can contain more than one extrusion die. The apparatus can also contain a hopper or container for the scaffold composition and a feeding system that is connected to the extrusion unit. A pump can be connected to the extruder as well, such that the scaffold composition is forced to and through the extrusion die.

Extrusion dies of different shapes can deposit the scaffold composition as different patterns, orientations, or thicknesses. The extrusion dies can be circular, flat, singular capillary. Any combinations of these dies can be used. The extrusion unit can also contain interchangeable dies.

The extrusion unit can be a standard apparatus configured to deposit compositions onto a surface (printing surface). The printing surface can be below (under) the extrusion unit, such that the scaffold compositions are forced downward onto the printing surface. Alternatively, the extrusion unit can be adjacent to the printing surface such that the scaffold compositions are forced laterally on to the printing surface. The extrusion unit can be connected to the X-Y-Z movement system such that the extrusion unit can be moved along the X, Y, and/or Z plains when in use.

The extrusion unit can also be operably connected to a temperature control device to heat or cool the scaffold compositions. The extrusion unit can also be operably connected to a pressure control device to control the pressure and/or volume at which the scaffold compositions are forced through the extrusion die.

The printing platform of the apparatus can support the printing surface. The platform can be connected to the X-Y-Z movement system such that the platform can be moved along the X, Y, and/or Z plains when in use.

In some examples, the platform can be perforated and connected to a suction device such that a suction is pulled through the platform. In this way the scaffold compositions can be drawn to the printing platform, and thus printing surface, by the suction.

In still other examples, the printing platform can be substantially flat. In other examples, the printing platform can be tubular. In other examples, the printing platform can have 6 degrees of freedom. In other examples, the printing platform can be a preform, which is shaped like a desired article. The preform can be shaped like a portion of a body.

The flat collection system can be a micro-perforated plate mounted on a CNC stage. The X-Y movement control the positioning and orientation of the fiber deposition. The Z axis can be used to set the Die to Collector Distance (DCD) and maintain it, as more layers are added. The microperforations are for the suction. The system works in a similar manner to additive layer printers with the difference that scaffold composition is the material deposited in layers over a controlled area and shape. There is not only control over the local thickness but also on the local orientation. The collection plate could be textured or have relief, to non-flat fabrics.

The tubular or cylindrical collection system can be a rotating micro-perforated collapsible cylinder. The cylinder also moves along its axis, to expose its entire length to the die. The ratio of the translational speed to the rotational speed controls the angle of scaffold composition deposition. Changing the die size allows control of the scaffold diameter of a wider range than customary. Selective deposition allows shapes like dumbbell in addition to regular cylinders.

The 6 degrees of freedom collection system can be a robotic arm holding a micro-perforated collapsible three-dimensional shape. This allows the same degree of control and texture as the flat collector but over a spheroid surface. The shape could be a shoe or a bladder (for organ manufacture).

The distance between the extrusion die and the printing surface can be varied, depending on the scaffold structure. Generally, shorter distances result in narrow and thick layers of scaffold composition, whereas longer distances result in broad thin layers of scaffold composition. The force at which the scaffold composition passes through the extrusion die also effects the structure. Generally, high pressures result in broad thin layers of scaffold composition and low pressures results in narrow and thick layers. The temperature of the extrusion unit or die can be varied to facilitate the printing. The choice of temperature can be made based on the type of scaffold composition being printed. The choice of die can also affect the scaffold structure. Capillary dies offer fine resolution and flat and circular dies offer coarser resolution.

The X-Y-Z movement system can be any system that can move the extrusion unit and/or printing platform in the X, Y, and Z directions. Such systems can be commercially available such as the CNC router type system, a 6 degree of freedom robotic arm, or rotating mandrel. The X-Y-Z movement system can be operably connected to one or more computers that control the movement in the X, Y, and Z directions based on coordinates inputted into the computer.

Kits

Kits for using the disclosed scaffold compositions and for practicing the disclosed methods are further provided. By “kit” is intended any manufacture (e.g., a package or a container) comprising at least one reagent, e.g., anyone of the compounds described herein. The kit may be promoted, distributed, or sold as a unit for performing the methods of the present invention. Additionally, the kits may contain a package insert describing the kit and methods for its use. Any or all of the kit reagents may be provided within containers that protect them from the external environment, such as in sealed containers or pouches.

To provide for the administration of such dosages for the desired therapeutic treatment, in some embodiments, pharmaceutical compositions disclosed herein can comprise between about 0.1% and 45%, and especially, 1 and 15%, by weight of the total of one or more of the compounds based on the weight of the total composition including carrier or diluents. Illustratively, dosage levels of the administered active ingredients can be: intravenous, 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasal instillation, 0.01 to about 20 mg/kg; and aerosol, 0.01 to about 20 mg/kg of animal (body) weight.

Also disclosed are kits that comprise a composition comprising a compound disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those agents described herein. In one embodiment, a kit includes instructions or packaging materials that describe how to administer a compound or composition of the kit. Containers of the kit can be of any suitable material, e.g., glass, plastic, metal, etc., and of any suitable size, shape, or configuration. In one embodiment, a compound and/or agent disclosed herein is provided in the kit as a solid, such as a tablet, pill, or powder form. In another embodiment, a compound and/or agent disclosed herein is provided in the kit as a liquid or solution. In one embodiment, the kit comprises an ampoule or syringe containing a compound and/or agent disclosed herein in liquid or solution form.

### EXAMPLES

The following examples are set forth below to illustrate the methods, compositions, and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

All chemicals were purchased from Sigma Aldrich (St. Louis, Mo.) unless otherwise indicated. Chitosan (practical grade, >75% deacetylated, MW=190,000−375,000 Da), hyaluronic acid (hyaluronic acid sodium salt, from Streptococcus equi), acetic acid (ReagentPlus, ≥99%), and hexamethyldisilazane (HMDS) were used as received, alginate (from brown algae), potassium bromide (FTIR grade, ≥99%, MW=11900 g/mol), Triton X-100 and Alizarin Red were used as received. Dulbecco's Modified Eagle Medium (DMEM) with 4.5 g/L glucose and L-glutamine without sodium pyruvate was purchased from Cellgro (Corning, N.Y.). Hyclone Non-Essential Amino Acids (NEAA) were purchased from GE Lifesciences (Pittsburgh, Pa.). Roswell Park Memorial Institute (RPMI) 1640 medium, Ham's F-12K medium with, Penicillin-Streptomycin, Dulbecco's phosphate buffered saline (D-PBS), 0.25% Trypsin-EDTA and Alamar blue reagent were purchased from ThermoFisher (Carlsbad, Calif.). Accumax was purchased from Innovative Cell Technologies (San Diego, Calif.). Fetal bovine serum (FBS) was purchased from Atlanta Biologicals (Flowery Branch, Ga.). Human PCa cell lines PC-3, C4-2B and 22RV were purchased from American Type Culture Collection (ATCC, Manassas, Va.) and were maintained according to manufacturer's instructions. PC-3 cells were cultured in F-12K, C4-2B and 22RV cells were cultured in RPMI 1640, with both media containing 10% FBS and 1% penicillin-streptomycin, at 37° C. and 5% CO₂ in a fully humidified incubator.

### Mechanical Characterization

Mechanical properties of scaffolds were obtained through compression testing of dry and wet samples (n=10 per group). The scaffolds were cut into 10 mm×10 mm×10 mm or 10 mm×10 mm×5 mm cubes and the samples were compressed at rate of 0.4 mm/min to 40% of initial height by AGS-X mechanical tester with 500 N load cell (Shimadzu, Japan). Wet samples were prepared by neutralizing (C-HA or C-CS) or crosslinking (CA) the cut samples with 1 M sodium hydroxide solution and washed with deionized water to remove any residual base. The wet samples were crosslinked, washed with D-PBS, and stored in D-PBS until tested. The Young's modulus and compressive strength of the scaffolds were determined from the resulting stress-strain plots and the average values were calculated from the ten samples.

### FTIR Analysis

The scaffolds were assessed with Fourier Transform Infrared (FTIR) spectroscopy to evaluate the polyelectrolyte complex (PEC) formation. The scaffolds were cut into very fine pieces, then mixed and ground with potassium bromide. The mixed power was compressed to 1 mm thickness semi-transparent pellet with a Quick Press KBr Pellet Kit (International Crystal Lab, Garfield, N.J.). The samples were analyzed with FTIR (Nexus 870, Thermo Nicolet, Waltham, Mass.) with 1000 scans at 8 cm⁻¹ resolution and compared to pure chitosan and alginate samples.

### Porosity Analysis

Scaffold porosity was measured using a modified liquid displacement method where isopropanol was used as the displacement liquid. Rectangular samples (10 mm×10 mm×5 mm, n=5) for each scaffold composition were prepared, the sample dimensions were measured using caliper, and the volume (Vi) of the scaffold was calculated. The weight (Wᵢ) of the dry scaffold was measured using an electronic analytical balance (ML54T, Mettler Toledo, Switzerland). The scaffold was fully immersed in 30 mL of isopropanol of known density (ρᵢ=0.785 g/mL) under vacuum for 30 min to dispel air from the pores. The isopropanol saturated scaffold was weighed (W_{f}) and the porosity was calculated (Eqn. 1).

$$Porosity = \frac{( {W}_{f} - {W}_{i} ) / {ρ}_{i}}{{V}_{i}} \times 100 \%$$

### Pore Size Measurement

The scaffold pore size was analyzed using scanning electron microscopy (SEM) images at 50× magnification. A template of 5 parallel lines spanning the image with 18 mm spacing was used. The number of pores intersected by the lines were counted and the length of line was divided by the number of pores to quantify the pore size for each scaffold. The line length was converted to μm using the scale bar in the SEM images. An average pore size was calculated from the results of the five lines for three images per scaffold group.

### Viscosity Measurement

The viscosity of the C-HA solutions was measured using a Haake Viscotester iQ Rheometer (HAAKE) with an electronically commutated motor system. The measuring geometry used was CC25 DIN/Ti. The samples were tested at 25° C. maintained by a Peltier cylinder temperature module with heat exchanger iQ (HX iQ, HAAKE) and measured with increasing shear rate over time for 500 s to a shear rate of 100/s.

### Cell Seeding

Cells (e.g., PC-3, C4-2B, 22RV, and MDA-MB-231-GFP cells) were expanded in T-75 cell culture flasks with containing 10% FBS, 1% pen/strep, and 1% NEAA to obtain the required cell number. The cells were detached from the culture flasks with Trypsin-EDTA, pelleted, and resuspended in fully supplemented media at 1,000,000 cells/ml. The cells were seeded on CA scaffolds in 100 μL of fully supplemented media and cultured in tissue culture plates. The seeded samples were incubated at 37° C. and 5% CO₂ for 1.5 h then 1.5 ml of fully supplemented media was added. The cell seeded scaffolds were transferred to new plates after 48 h to avoid any effects from cells attached to the bottom of the well. The samples were cultured for 15 d and media was changed every other day.

### Cell Proliferation Assessment

The cell number with respect to time was analyzed with AlamarBlue assay at 5 d, 10 d, and 15 d timepoints. The media was aspirated and the wells were washed with D-PBS. A 10% AlamarBlue solution was prepared by adding AlamarBlue reagent into fully supplemented media and 1 mL of 10% AlamarBlue solution was added to each well of 2D samples and 3D scaffolds. The samples were incubated at 37° C. and 5% CO₂ for 1.5-2 h, then the AlamarBlue solution was transferred to a black 96-well plate. Fluorescence measurement was performed using a Cytation5 multi-mode imaging microplate reader (Biotek, Winooski, Vt.) at excitation wavelength of 560 nm and fluorescence emission wavelength of 590 nm or 595 nm. The cell number was calculated based on a standard curve generated from a series known cell numbers of 231-GFP cells grown as monolayers.

### Scanning Electron Microscopy Analysis and Energy Dispersive Spectroscopy

The 2, 4 and 6 wt % CA scaffolds were sputter coated with Pd—Au and imaged with JSM-6480 SEM (JEOL, Japan). Scaffold cultured samples were fixed with 3.7% formaldehyde in D-PBS for 1 h at 37° C. at 5 d, 10 d, and 15 d. The samples were washed with D-PBS and stored in D-PBS at 4° C. until dehydration. The fixed samples were dehydrated with graded ethanol washes (0%, 30%, 50%, 75%, 85%, 95% and 100%) with each ethanol concentration applied three times for 5 min each time. After washing with 100% ethanol, samples were either washed with HMDS or freeze dried overnight to yield dry samples. The HMDS treated samples were washed twice with a 1:1 solution of HMDS and 100% ethanol, then washed with 100% HMDS once, and placed in 100% HMDS to air dry for 12 h in a fume hood. The samples sputter coated with Pd—Au and imaged with the same JSM-6480 SEM. While taking SEM images, energy dispersive spectroscopy (EDS) was applied to the individual cell surface to detect the elements on the selected cell surface. EDS characterized the elemental composition of selected cells with 2000 scans at 15 kV and 60 μm aperture.

### Immunofluorescence Sectioning Imaging

The formaldehyde fixed samples were cut into half perpendicular to the flat orientation and mounted in paraffin. The samples were sectioned to 5 μm thickness, mounted on slides, and the paraffin was removed through heating and washing with xylene and ethanol. The samples were rehydrated in TE buffer. 2D cultured cells on coverslip was fixed in formaldehyde and washed with PBS then permeabilized by 0.1% Triton X-100 for 15 minutes. The C4-2B and 22RV samples were assessed with primary antibodies including cytokeratin 8 (diluted in 10% BSA with ratio of 1:100) (Santa Cruz Biotechnology, Santa Cruz, Calif.), rabbit mAb androgen receptor (diluted in 10% BSA with ratio of 1:50) (Cell Signaling Technology, Danvers, Mass.), and anti-osteocalcin antibody (diluted in 10% BSA with concentration of 10 μg/ml) (Abcam, Cambridge, Mass.). The PC-3 samples were assessed with primary antibodies including rabbit monoclonal phospho-EGF receptor (diluted in 10% BSA with ratio of 1:500) (Cell Signaling Technology, Danvers, Mass.), anti-osteocalcin antibody, and cytokeratin 8. The primary antibodies were incubated overnight at room temperature in a sealed box to avoid evaporation. The samples were washed with D-PBS, then the secondary antibodies were added. The secondary antibodies included DyLight 488 horse anti-mouse IgG antibody, biotinylated horse anti-rabbit IgG antibody, and Texas Red Streptavidin (all diluted in 10% BSA with ratio of 1:200) (all from Vector Laboratories, Burlingame, Calif.). The secondary antibodies were incubated for 20 min then washed with D-PBS, with the biotin and streptavidin antibodies being applied in two incubations. The samples were mounted with DAPI Fluoromount-G (SouthernBiotech, Birmingham, Ala.) and a coverslip on the section. The sections were imaged with a Leica TCS SP8 confocal microscope by 405 diode, argon and DPSS 561 laser. (Leica, Buffalo Grove, Ill.).

### Alizarin Red Staining

The same paraffin blocks used for immunofluorescence staining was sectioned to 15 μm thickness, mounted on slides, and the paraffin was removed through heating and washing with xylene and ethanol. Around 100 μl of 2 wt % alizarin red solution with pH of 4.2 was dropped to the sample area and incubate in dark under room temperature for 2 minutes. Rinse with DI water after incubation, then keep in acetone for 20 seconds, acetone-xylene (1:1) solution for 20 seconds. Clear in xylene for 5 minutes and mount a coverslip with mounting media.

### Real Time-qPCR Assessment

Cells were harvested from the scaffolds at 5 d, 10 d, and 15 d. Samples were transferred into a new 12 well plate to avoid the cells attached to the well bottom. Then, added 2 ml Accumax to each sample and shaken for 15 mins. After shaking, washed with the existing Accumax by micropipette for a few times and collected all the Accumax solution in a tube and added another 2 ml of Accumax to shake the sample for another 15 mins. Then washed by micropipette with the existing Accumax and collected the solution in the same tube. The cell solution was spun down by centrifuge for qPCR use. The RNA extraction was performed with Qiagen RNeasy Kit (Qiagen, Netherlands). The primers and internal control applied were AR, PSA, EIF3D and RPL13A (Qiagen, Netherlands) and the samples were incubated at 37° C. for 1 h and 95° C. for 5 min in GeneAmp PCR System 9600 (Perkin Elmer, Waltham, Mass.) for cDNA synthesis. The qPCR assessment was performed with the Rotor-Gene Q (Qiagen) with the following conditions: 95° C. for 15 min, 40 cycles at 94° C. for 15 s for renaturation, 55° C. for 30 s for annealing and 72° C. for 30 s for extension.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1: Chitosan-Alginate (CA)

Chitosan solutions were prepared by dissolving chitosan (2 wt %, 4 wt %, and 6 wt %) into acetic acid solution (0.5 wt %, 1 wt %, and 1.5 wt %) and mixing with a Thinky mixer (ARM-310, Thinky USA) at 2000 rpm for 3 min. Alginate solutions (2 wt %, 4 wt %, and 6 wt %) were prepared by dissolving alginate in DI water and mixing with a Thinky mixer at 2000 rpm for 3 min. The solutions were aged at room temperature overnight, then the chitosan and alginate solutions were stirred together and mixed with a Thinky mixer at 2000 rpm for 5 min twice. The CA solution was cast into molds, refrigerated at 4° C. for 1.5 hours, then frozen at −20° C. overnight and lyophilized for 48 hours. The CA scaffolds were cut into 2 mm thick discs and cross-linked with 0.2 M calcium chloride solution for 15 min under vacuum. The scaffolds were washed with DI water at least 4 times to remove any residual calcium. The scaffolds were then sterilized with 70 vol % ethanol under vacuum for 15 min twice, followed by washing with excess PBS three times and shaking with excess D-PBS overnight to prepare the scaffolds for in vitro trials.

The CA scaffold properties were characterized with mechanical testing in dry and wet conditions, scanning electron microscopy (SEM) imaging, and FTIR spectroscopy. C4-2B, 22Rv1, and PC-3 cells were seeded on CA scaffolds and 2D surfaces. The media used was RPMI 1640 medium with L-glutamine, 10% FBS, and 1% pen/strep for C4-2B and 22Rv1 cells and Ham's F-12K medium with L-glutamine, 10% FBS, and 1% pen/strep for PC-3 cells. All samples were cultured in a humidified incubator at 37° C. and 5% CO₂ for 15 days. The in vitro samples were characterized at 5 d, 10 d, and 15 d timepoints with AlamarBlue assay, SEM, immunofluorescence, and qRT-PCR.

3D porous CA scaffolds were produced with different concentrations to yield scaffolds that mimicked the stiffness of PCa tumor microenvironment conditions reflecting normal prostate tissue, primary PCa tumor, and bone metastatic microenvironment. The 2 wt % CA scaffolds were white, while the 4 and 6 wt % CA scaffolds were a tan color. The CA scaffold compositions had similar porosities: 90.78±0.6%, 93.16±2.2%, and 91.44±2.0% porosity for the 2, 4, and 6 wt % CA scaffolds, respectively. The pore morphology for all three CA scaffold compositions was evaluated with SEM imaging (FIG. 2). The 2, 4, and 6 wt % CA scaffolds have similar pore sizes: 253±52 μm, 210±21 μm and 217±33 μm, respectively (Table 1).

TABLE 1
Porosity and pore size for CA scaffolds.

| | Porosity | Pore Size (μm) |
|---|---|---|
| 2 wt % CA | 90.78% ± 0.65% | 253 ± 52 |
| 4 wt % CA | 93.16% ± 2.2% | 210 ± 21 |
| 6 wt % CA | 91.44% ± 2.1% | 217 ± 33 |

The scaffold porosity was formed through the freeze casting process, where the CA solution was frozen, causing ice crystals to nucleate and grow throughout the solution. The samples were lyophilized to sublimate the ice crystals and a porous scaffold with interconnected pores remained. The pore morphology and pore size were influenced by the nucleation rate and the size of ice crystals, which depend on the rate of heat diffusion from freezing environment to the center of cast solution. Pore size and percent porosity influences cell viability and proliferation in tissue engineering scaffolds, with pore sizes between 200 μm to 250 μm and greater than 85% porosity promoting cell proliferation. Therefore, all CA scaffold compositions should be suitable for cell growth.

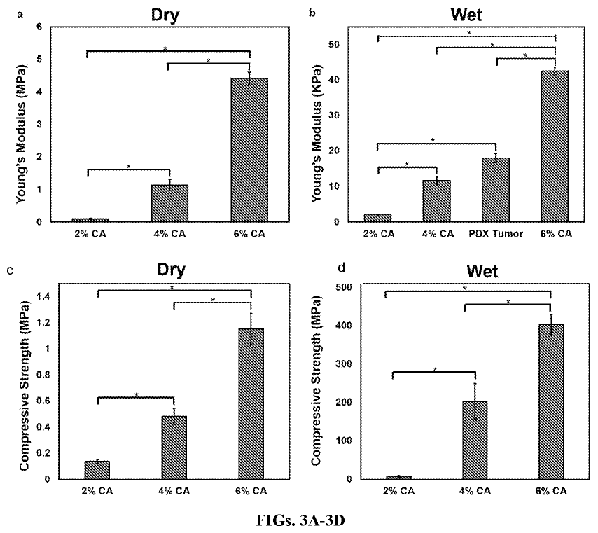

The compressive stiffness (FIGS. 3A and 3B) and compressive strength (FIGS. 3C and 3D) of the three CA scaffold compositions were measured in dry and wet states. The stiffness of the dry 2, 4, and 6 wt % CA scaffolds is 0.097±0.017 MPa, 1.13±0.17 MPa, and 4.42±0.19 MPa, respectively. The stiffness of the wet 2, 4, and 6 wt % CA scaffolds is 2.1±0.11 kPa, 11.7±1.10 kPa, and 42.5±1.03 kPa, respectively. The compressive strength of the dry 2, 4, and 6 wt % CA scaffolds was 0.138±0.013 MPa, 0.483±0.061 MPa, and 1.154±0.119 MPa, respectively. The compressive strength of the wet 2, 4, and 6 wt % CA scaffolds was 7.75±2.32 kPa, 203.31±46.63 kPa, and 403.13±25.44 kPa, respectively. The increased scaffold concentrations from 2 to 6 wt % CA led to a significant increase in scaffold stiffness and compressive strength in both dry and wet conditions (FIGS. 3A-3D). The stiffness of PCa PDX tumors were measured with the same method and was 17.97±1.29 kPa, indicating that the wet 4 wt % CA scaffolds most closely approximated the PCa tumor stiffness out of the three CA scaffold compositions.

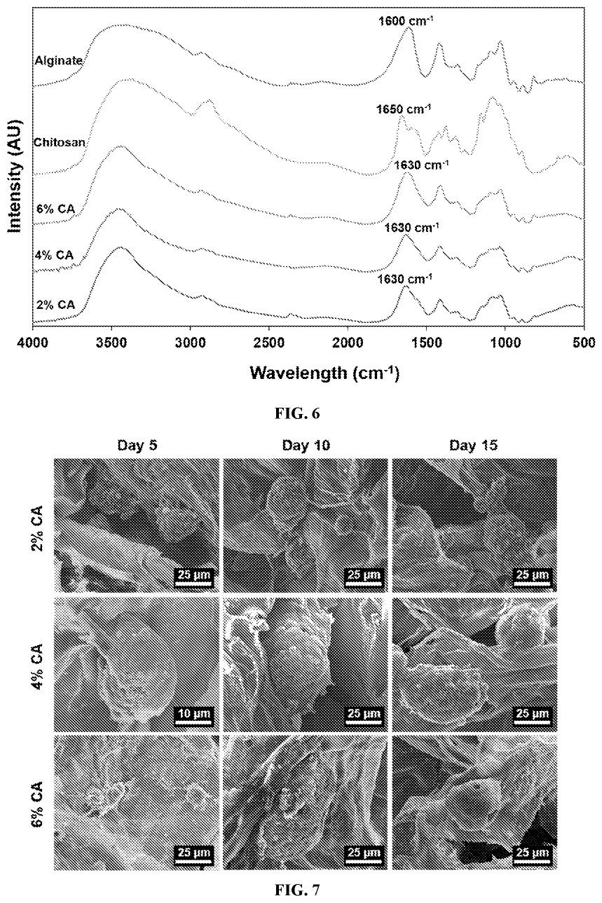

FTIR analysis was performed to assess the PEC formation in the CA scaffolds. The FTIR spectra for the CA scaffold compositions, chitosan, and alginate are shown in FIG. 6. Characteristic peaks in the alginate spectra include the peaks at 1600 cm⁻¹ (COO⁻ antisymmetric stretch), 2920 cm⁻¹ (C—H stretch), and 3430 cm⁻¹ (O—H stretch). Characteristic peaks in the chitosan spectra includes the peaks at 2900 cm⁻¹ (C—H stretch), 1650 cm⁻¹ (amide I, C═O), and 1590 cm⁻¹ (amide II, N—H). The chitosan used for scaffolds synthesis is partially N-deacetylated 75% deacetylated) which causes the appearance of double amide peaks (1650 cm⁻¹ and 1590 cm⁻¹). CA PEC formation is indicated by the combination of amide I (1650 cm⁻¹) and N—H bending of amine II (1590 cm⁻¹) chitosan peaks and the COO⁻ antisymmetric (1600 cm⁻¹) alginate peak into one peak at 1630 cm⁻¹, which is shown on the 2, 4, and 6 wt % CA spectra.

Three PCa cell lines, PC-3, C4-2B and 22Rv1, were cultured on the three CA scaffold compositions. The three PCa cell lines have different levels of malignancy, with PC-3 cells being derived from a lumbar metastasis, C4-2B cells derived from a bone metastasis of the LNCaP parental line and 22Rv1 cells derived from human prostatic carcinoma xenograft, CWR22R. The CA scaffold cell number was assessed at 5, 10, and 15 d using the Alamar Blue assay. At 5 d, the 2 wt % CA scaffolds had the greatest cell number and 6 wt % CA had the lowest cell number for all three cell lines. At 10 d, 2 wt % CA had the greatest cell number for PC-3, 4 wt % CA had the greatest cell number for C4-2B and 6 wt % CA had the greatest cell number for 22Rv1. At 15 d, 6 wt % CA has the greatest cell number for all cell lines. It appears that all cell lines reach a maximum cell number at 10 d, then decrease in cell number. This could be due to several factors, including: necrotic cells in spheroids; metabolic reprogamming; or other factors. The cell number was only proportional with CA scaffold stiffness at 15 d, where 6 wt % CA has greatest cell number, followed by 4 wt % and 2 wt % CA for all three cell lines. This result needs further investigation to examine the cell number over the greater culture times and use other cell number assays for orthogonal measurements. Other than at 15 d, there were no clear trends in the PCa cell growth with scaffolds stiffness. Similar results for PC-3 and 22Rv1 cells have been observed in 2D cultures, indicating that the growth of these two cell lines was not influenced by the matrix stiffness.

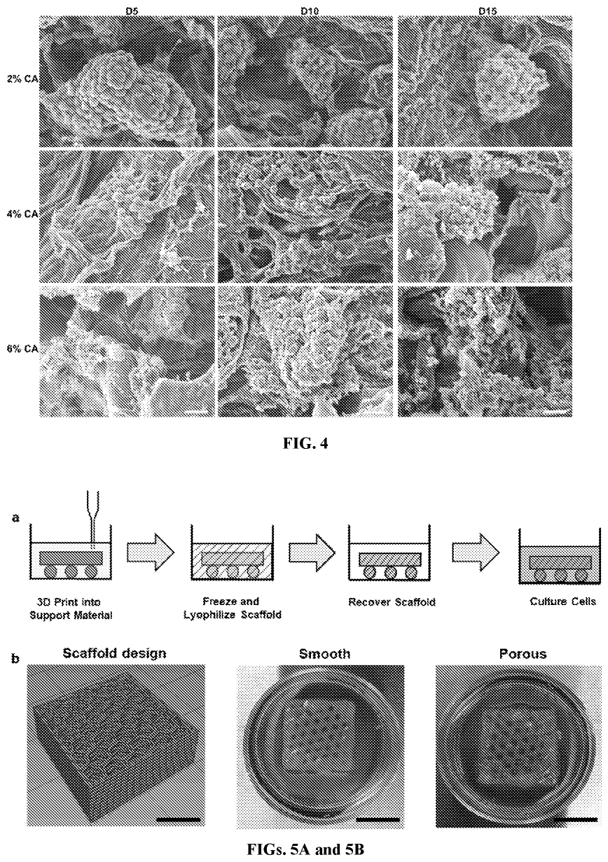

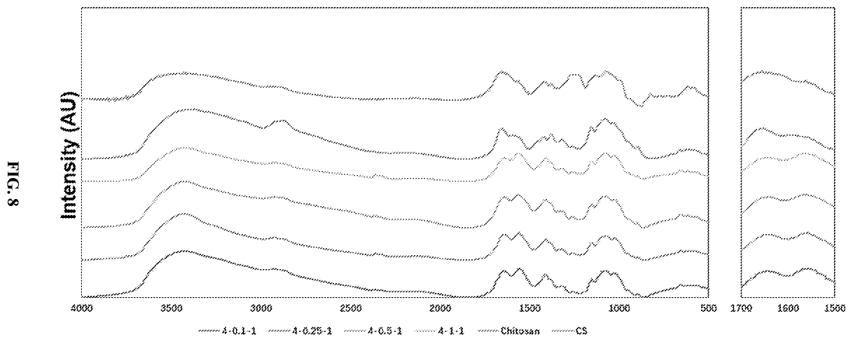

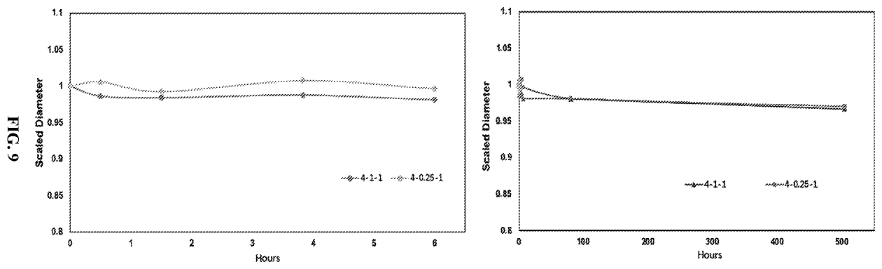

The cell morphology of the three PCa cell lines on CA scaffolds at 5, 10, and 15 d is shown in FIGS. 2, 4, and 7. All three cell lines had a rounded morphology on the 2, 4, and 6 wt % CA scaffolds. Despite the common rounded morphology, there were differences in the cell appearance in the CA scaffold cultures. The PC-3 cells formed cell clusters that covered the whole scaffold surface in 15 d, while C4-2B and 22Rv1 cells formed multicellular spheroids. C4-2B cells had formed spheroids by 5 d. 22Rv1 also formed spheroids, which were present in 2 and 4 wt % CA scaffolds at 5 d. The 22Rv1 spheroids had holes that formed on the outside of the spheroids (indicated with white arrow) as shown at 15 d on 6 wt % CA. Based on these images, the PC-3 cell morphology was not affected by the CA scaffold compositions. 22Rv1 cells on 6 wt % CA at d 10 and d 15 had much smoother spheroid surfaces than 2 and 4 wt % CA, which made the cell-cell adhesions much harder to visualize. This appears to be additional extracellular matrix secreted by the cells. The C4-2B and 22Rv1 spheroid surfaces had an increasing rough appearance with increasing culture time from 5 to 15 d, with a greater effect noted in the C4-2B cultures. The C4-2B cells grown on 6 wt % CA had much rougher spheroid surface than those in 2 and 4 wt % CA at 10 d. The PC-3 cultures did not demonstrate the rough appearance, instead maintaining a smooth cell surface during the culture. We hypothesized that this rough appearance is mineralization on the PCa spheroids.

Immunofluorescence staining was used to evaluate the malignancy of the CA scaffold cultures by evaluating the presence of phospho-epidermal growth factor receptor (pEGFR), androgen receptor (AR), and cytokeratin 8 (KRT8) in the samples. KRT8 was examined in all three cell lines, but AR was only examined in C4-2B and 22Rv1 as PC-3 has a truncated AR. PC-3 was examined with pEGFR in place of AR. KRT8 and pEGFR were expressed in all PC-3 cultures. The 4 wt % CA cultures qualitatively had the greatest expression of pEGFR while 6 wt % CA cultures had lowest. However, the expression of KRT8 of PC-3 cells on 6 wt % CA was qualitatively greater than the other two compositions. The pEGFR expression in the 2D samples was observed in the nucleus, while the scaffold cultured PC-3 cells had expression in the cell membrane. C4-2B cells grown on 2D and all CA scaffold compositions expressed AR and KRT8. The cells cultured on 2 wt % CA qualitatively had the greatest expression of both AR and KRT8. AR and KRT 8 were also expressed on 22Rv1 cells grown on 2D and CA scaffolds. The AR and KRT 8 expression of cells on 4 wt % CA was qualitatively greater than the other CA scaffolds and 2D. KRT8 is an intermediate filament protein found in normal prostate epithelial cells, but also can be found on cell membrane of some carcinoma cells. Androgen is an important factor that stimulates PCa cell proliferation and prevents apoptosis. Therefore, AR is commonly used to characterize PCa cells. EGFR is a member of ErbB family of tyrosine kinase receptors which can facilitate cancer cell growth, metastasis, and invasion. It is regulated in normal cells but constantly stimulated in tumor cells. EGFR overexpression was shown to be correlated with the hormone refractory PCa phenotype and 17 out of 19 androgen independent PCa patients had increased EGFR expression.

Osteoblastic and osteoclastic responses of PCa cells have been demonstrated both in vivo and in vitro. This is due to various factors produced by PCa cells, like BMP-6, VEGF and semaphorin 3A, that can indirectly or directly induce an osteogenic response. To assess if the observed rough appearance of the cell spheroids was mineralization, additional characterizations, including Alizarin Red staining, EDS, and osteocalcin immunostaining, were performed to demonstrate the presence of calcium phosphates and bone matrix proteins. Alizarin Red staining is commonly used to indicate the presence of calcium deposits and is used to verify the osteogenic differentiation of mesenchymal stem cells. Positive Alizarin Red staining in C4-2B and 22Rv1 samples was observed, while no positive staining was observed for PC-3. The faint red color observed in 2, 4 and 6 wt % scaffolds of PC-3 cells and also present in regions of the other cell line samples is believed to be background staining of the scaffold, not positive staining for mineralization.

While the Alizarin Red staining denoted the presence of calcium phosphate mineralization, in vitro cultures are prone to pathological calcification, so additional characterizations were used to confirm the presence of bone mineral. EDS was used to evaluate the 10 d samples for each cell line to quantify the amount of calcium and phosphorous elements, which are two elements found in bone mineral. All cell lines had calcium and phosphorus elements on the cellular surfaces. While all cell lines and scaffold compositions demonstrated the presence of calcium and phosphorus at 10 d, the location of these elements is unclear since EDS has a penetration depth that will go within the cell. If intracellular composition was detected, it could explain the presence of calcium and phosphorus in the PC-3 samples, while no mineralization was observed in the SEM samples or Alizarin Red staining.

Finally, to confirm that the observed mineralization was bone mineral and not pathological calcification, osteocalcin expression was assessed with immunofluorescence. Osteocalcin is a non-collagenous protein secreted by osteoblasts that is found in bone matrix. Osteocalcin plays a role bone mineralization and is important for the physical and chemical properties of bone. The osteocalcin expression for PC-3, C4-2B and 22Rv1 cell lines on 2, 4, and 6 wt % CA scaffolds demonstrated that C4-2B and 22Rv1 cultures expressed osteocalcin while PC-3 did not. This confirms the presence of mineralized bone matrix for C4-2B and 22Rv1 cultures, while indicating that PC-3 cultures did not exhibit any bone matrix, despite positive EDS results for calcium and phosphorus. Osteocalcin expression demonstrates the presence of bone matrix and further supports that the mineralization observed with SEM and Alizarin Red analysis is bone mineral and not pathological calcification.

These mineralization results agree with the literature. PC-3 cells were shown to induce osteoblast-like cells to express osteoclastogenesis factors after co-culture or when cultured with PC-3 conditioned media. The osteolytic behavior of PC-3 cells was demonstrated by injecting PC-3 cells into implanted humanized tissue engineered bone constructs in mice, where the bone was resorbed. Alternatively, C4-2B cells enhanced the mineralization of hematopoietic progenitor cells. C4-2B cells were shown to mineralize in 2D cultures with osteogenic media (containing 10 mM β-glycerophosphate and 50 mg/ml L-ascorbic acid), providing strong support for our hypothesis about C4-2B mineralization. Our results are exciting as these cultures were conducted in basal media without any osteogenic supplements; including these supplements would likely enhance the mineralization in the samples.

The gene expression for all three cell types were evaluated at 10 d with qPCR. PSA and AR mRNA for the three PCa cell lines were tested to evaluate the PCa phenotype, with increased expression denoting a more aggressive phenotype. PSA is one of the most common markers to detect PCa malignancy. PSA expression could be related to PCa progression by downregulating cell apoptosis and increasing cell proliferation. All cell types on CA scaffolds had unregulated PSA expression compared with 2D cultures, especially the 22Rv1 cells on 6 wt % CA which had nearly a 3-fold increase of PSA expression compared to 2D. AR is another widely used marker for PCa. C4-2B on CA scaffolds all had lower AR expression than 2D while 22Rv1 cells all had greater AR expression than 2D. For 22Rv1 cells, the AR expression of cells grown on scaffolds increased with increasing stiffness. However, the AR expression of C4-2B cells grown on CA scaffolds decreased with increasing scaffold stiffness.

All PC-3 scaffold cultures had greater pEGFR expression compared with 2D. The 4 wt % CA cultures had the greatest expression of pEGFR among the scaffold cultures, while 6 wt % CA cultures had lowest expression, which was similar to the 2D cultures. The PC-3 scaffold cultures had similar KRT8 expression among the CA compositions, with all scaffold compositions showing KRT8 expression indicating the epithelial phenotype in cells in scaffold cultures. The C4-2B 2D cultures had the greatest AR expression and AR expression was downregulated in the scaffold cultures, with significant differences in expression between 2D and scaffold cultures. There was no significant difference in AR expression between the scaffold cultures. The scaffold cultures demonstrated KRT8 expression in all scaffold culture conditions indicating epithelial phenotype of C4-2B cells grown in in CA scaffolds. The 22Rv1 cultures had similar AR and KRT8 expression between the four groups. The 4 wt % CA scaffold cultures had the greatest AR expression compared to the other cultures. The KRT8 expression for 22Rv1 was relatively consistent among the cultures. The IF results show no significant changes in cell phenotype for PC-3 and 22Rv1, but a reduced AR expression in C4-2B cells in CA scaffolds.

The gene expression for all three cell lines and culture conditions was evaluated at 10 d with qRT-PCR to assess the preservation of PCa cell characteristics. C4-2B and 22Rv1 cell lines were evaluated for expression of AR and PSA mRNAs, while PC-3 was tested for expression of LIMK1 mRNA. One of the characteristics of C4-2B and 22Rv1 cell lines is that both cell lines express AR, which is a transcription factor. AR specifically stimulates transcription of PSA mRNA, hence expression of PSA represents the activation status of the AR. PSA is also the clinical gold standard biomarker for PCa diagnosis. PSA expression is a marker for PCa progression as PSA downregulates cell apoptosis and increases cell proliferation. LIMK1 is an enzyme that regulates the actin cytoskeleton and is overexpressed in PCa, specifically in PC-3 cells. LIMK1 is also critical for the PCa invasive growth. Because PC-3 cells are highly aggressive androgen-independent PCa cells, LIMK1 expression was used as a marker for aggressive phenotype of PCa cells.

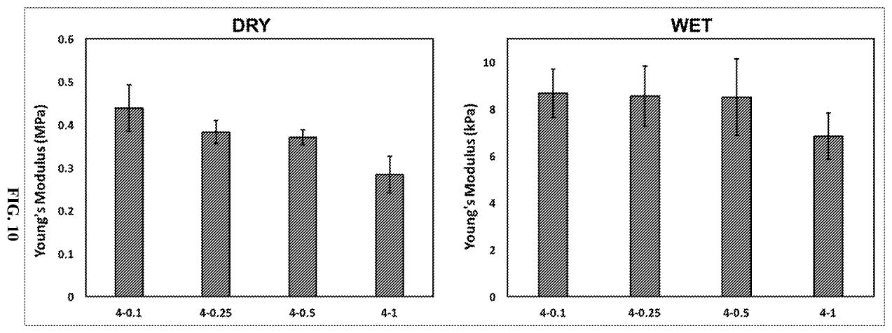

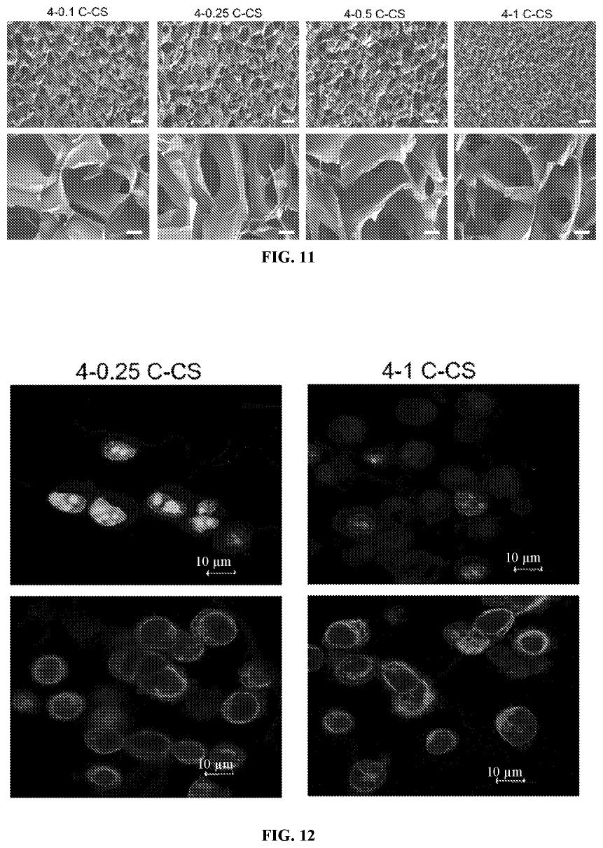

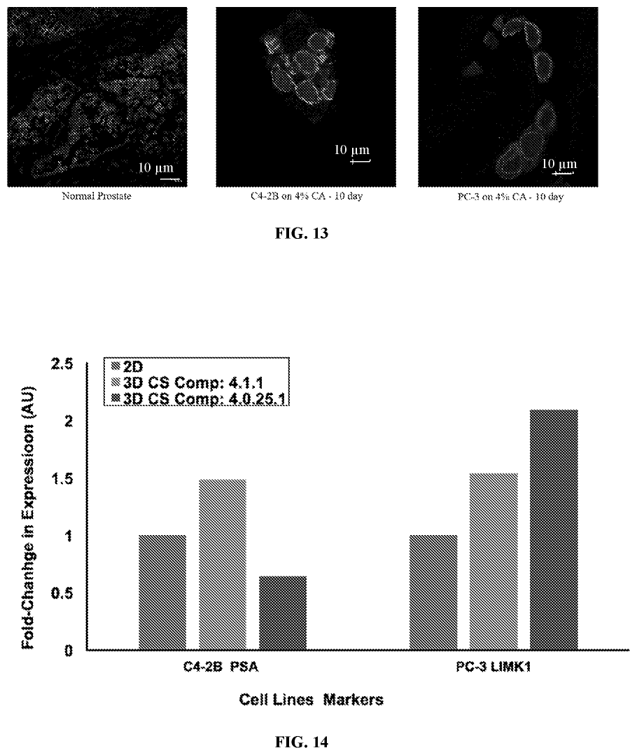

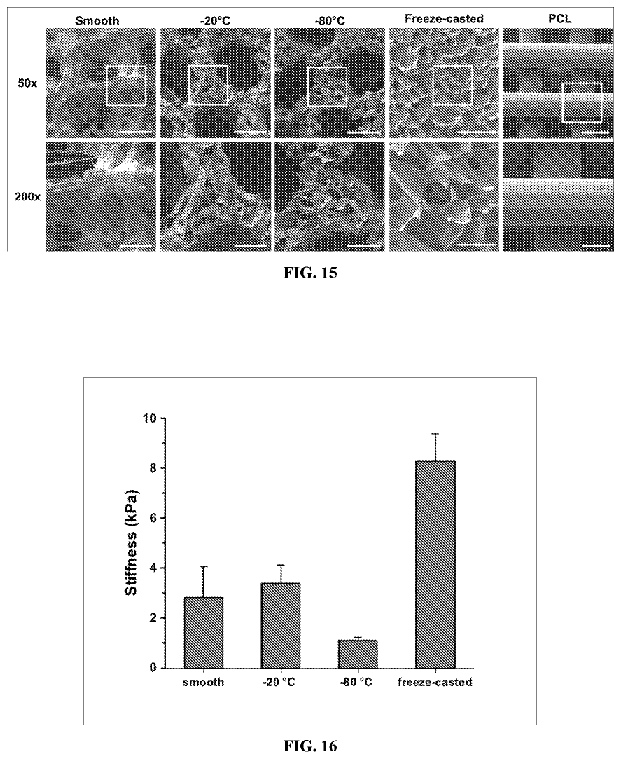

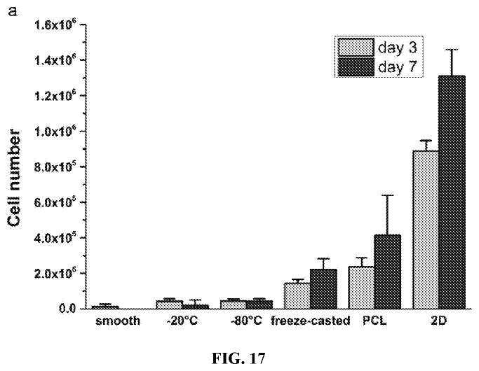

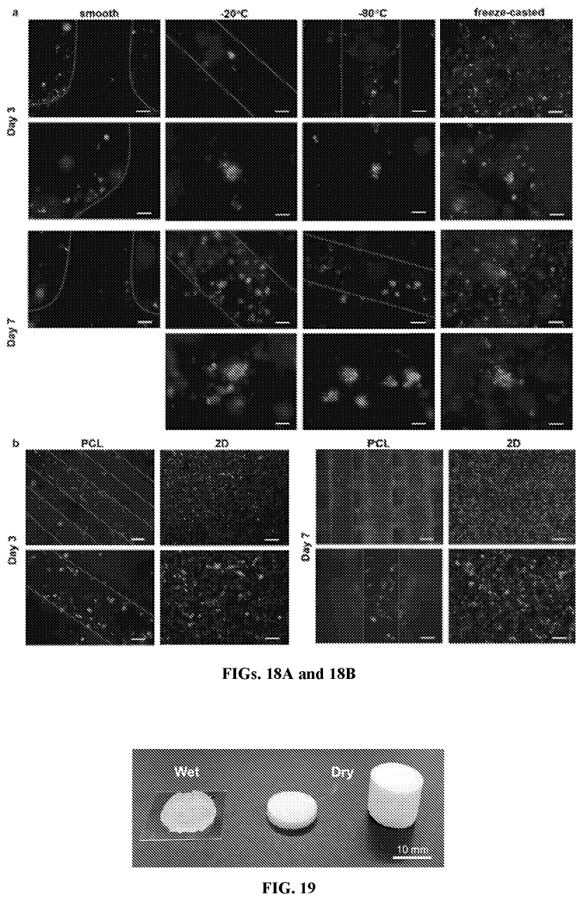

The PC-3 CA scaffold cultures had downregulated LIMK1 expression compared to the 2D cultures, with statistically significant differences between 2D and each scaffold composition. Among the scaffold cultures, LIMK1 expression showed a significant increase in 6 wt % cultures compared to 2 wt % cultures. PSA expression demonstrated different trends for C4-2B and 22Rv1 cultures with respect to stiffness (FIG. 10b). PSA expression did not change in C4-2B scaffold cultures at 2 wt % and 4 wt % CA compared with 2D cultures. The PSA expression for the 6 wt % CA cultures was significantly lower than the 2D and 2 wt % and 4 wt % CA scaffold cultures. All 22Rv1 CA scaffold cultures had upregulated PSA expression compared with 2D cultures with a maximum increase at 6 wt % CA compared to 2D cultures. Additionally, 22Rv1 cultures had statistically significant increase in PSA expression in 6 wt % CA compared to 2 wt % and 4 wt % CA scaffold cultures. There were no significant differences in AR expression observed for C4-2B and 22Rv1 scaffold cultures. The C4-2B AR expression was downregulated in the scaffold cultures compared to 2D cultures, whereas no significant change in AR expression in 22Rv1 cells was observed between 2D and 3D cultures. The C4-2B and 22Rv1 AR expression PCR results support the results of IF expression analysis. The marker expression analysis with IF and qRT-PCR demonstrated different responses to scaffold stiffness for each cell line. PC-3 cells demonstrated upregulation of pEGFR expression and downregulation of LIMK1 expression in the CA scaffolds. C4-2B cultures had downregulation of PSA and AR expression in 3D cultures, while 22Rv1 cultures had similar expression of AR between 2D and CA scaffold cultures and PSA expression was upregulated in the 6 wt % CA scaffolds. Collectively, the protein and gene expression results demonstrated that the CA scaffold cultures supported expression of phenotypic markers different expression profiles for each cell line.

Characterization of phenotype demonstrated a shift in marker expression for PC-3 cells grown in CA scaffolds. There was a loss of expression of LIMK1 but a gain in expression of pEGFR in PC-3 cells in 2 wt % and 4 wt % CA. Collectively, these observations suggest that PC-3 cells maintain their aggressive phenotype in 3D cultures. Similarly, there is no significant change in AR expression and activity in C4-2B and 22Rv1 cells grown in 2 wt % and 4 wt % CA scaffolds except PSA expression in 22Rv1 cells grown in 6 wt % CA scaffold culture. Similar PSA expression results for C4-2B and 22Rv1 cultures have been reported in the literature. Downregulation of PSA expression like in the C4-2B cultures was observed where LNCaP cells were cultured in rotary wall vessels as 3D organoids, but the PSA expression was restored when the 3D organoids were co-cultured with human prostate fibroblast cells. This demonstrates the importance of the stromal cell contribution to PCa malignancy that cannot be recreated with cultures only comprised of cancer cells. Upregulation of PSA expression like in the 22Rv1 cultures was observed where LNCaP cells cultured on collagen-based scaffolds had higher PSA expression compared with 2D cultures.

Based on the results from these studies, 3D porous CA scaffolds demonstrate potential for use as an in vitro assay or diagnostic to evaluate PCa malignancy and to evaluate potential chemotherapies. Such an in vitro diagnostic could be used to supplement pathology data from tumor biopsies. The diagnostic would use primary PCa cells directly from patients and seed the cells on CA scaffolds to determine the expression of phenotypic markers, potential level of malignancy, and screen prospective chemotherapies.

### Example 2: Chitosan-Chondroitin Sulfate (C-CS)

C-CS scaffolds were developed to mimic the PCa TME. The C-CS scaffold process can be adjusted to vary the scaffold stiffness (Young's modulus), composition, and pore size. Data for C-CS scaffolds demonstrated the production of a porous scaffolds with different stiffness. The C-CS scaffolds were demonstrated to support PCa culture, indicated by formation of tumor clusters and increasing cell numbers during the 15-day culture. Additional analyses demonstrated that the C-CS and CA scaffolds promoted the expression of functional PCa markers, including androgen receptor and phospho-EGF receptor. Four types of C-CS scaffolds can be prepared to provide an array of stiffness and composition. This C-CS scaffold array can allow examination of the influences of stiffness and composition in the TME on the culture of PPCa cells.

The 3D cultures in biomaterial (C-CS) scaffolds can be made using PCa cell lines (C4-2B and 22RV) and patient derived xenograft (PDX) primary cells and can be compared the cultures to 2D controls (tissue culture polystyrene and films with same composition as scaffolds) to evaluate the suitability of 3D culture for recapitulating the PCa cell growth pattern. The cell response can be characterized to different scaffold conditions that reflect the stiffness of normal and malignant prostate tissue (<5 kPa), and bone metastatic microenvironment (>30 kPa).

The phenotypic characteristics of the tumor cells can be determined by assaying cell PCa markers, cell morphology in 2D and 3D, drug response to standard of care therapies (e.g., ADT, docetaxel, flutamide, Xtandi, Zytiga, and cabazitaxel) and microRNA expression using RNAseq. Patient PPCa cells that failed multiple therapies can be tested in 3D culture. For instance, PRJ-68T patient failed Zytiga, Xtandi, docetaxel, and cabazitaxel. The BROV media can be used for all cultures. Cell proliferation and cell morphology in 3D cultures can be examined as published (Kievit F M, et al., (2010) Chitosan-alginate 3D scaffolds as a mimic of the glioma tumor microenvironment. Biomaterials 31(22):5903-5910). For PDX cells, tumors can be grown in mice, excised and single cell suspensions can be made using DNase/collagenase. To mimic the TME, tumor and stromal cells from PDX tumor- and patient PPCa tissues can be sorted and combined separately for a mixed population (MP) of cells from each tumor source and grown in 3D cultures. Spheroids developed from MP cells can be used for cell proliferation, drug sensitivity, and IHC-based PCa marker expression analyses. Data can be compared between 2D and 3D cultures of cells from PDX model and from patient PPCa cells separately. Next, data from PDX cells grown in 3D culture can be compared to the patient PCa cells grown in 3D culture to evaluate recapitulation of PPCa phenotype in PDX cells and when cells are grown in 3D cultures. For RNAseq analysis, homogeneous tumor cells grown (PDX tumor cells, patient PPCa cells, and PCa cell lines) can be used for both 2D and 3D cultures. Expression of PCa markers in spheroids developed from PDX MP cells and patient PCa MP cells can be compared to the expression in original patient tumor using paraffin embedded tissue sections.

### Example 3: Chitosan-Hyaluronic Acid (C-HA)

C-HA scaffolds were prepared following previously published methods (Florczyk S J, et al. Porous chitosan-hyaluronic acid scaffolds as a mimic of glioblastoma microenvironment ECM. Biomaterials. 2013; 34:10143-50). The C-HA method was adapted by varying chitosan concentration (2 or 4 wt %), HA concentration (0.5, 1, and 2 wt %), and acetic acid concentration (0.5, 1, 2.5, 5, and 10 wt %) to yield seven compositions of C-HA scaffolds (Table 1). The naming convention used for the C-HA scaffold compositions consists of chitosan concentration—HA concentration—acetic acid concentration, i.e. 4-1-5 for 4 wt % chitosan, 1 wt % HA, and 5 wt % acetic acid. Briefly, equal volumes of chitosan solution and hyaluronic acid solution were prepared with the same acetic acid concentration and the solutions were mixed twice for three minutes each in a Thinky mixer (ARM-300, Thinky USA, Laguna Hills, Calif.). The solutions were aged at room temperature overnight, then stirred together and mixed twice for five minutes in the Thinky mixer to form C-HA polyelectrolyte complex (PEC). The C-HA solution was cast into 24-well plate molds, refrigerated at 4° C. for 1 h, then frozen at −20° C. overnight, and lyophilized with a freeze drier (Virtis Freezemobile) for 48 h to form a porous structure. After lyophilization, C-HA scaffolds were sectioned into 2 mm thick discs, neutralized with 1 M sodium hydroxide solution for 1 h under vacuum, and washed five times with excess DI water to remove any remaining base. The neutralized scaffolds for in vitro analysis were sterilized with 70% ethanol for 30 min under vacuum, and washed with D-PBS three times to remove remaining ethanol in a biosafety cabinet, followed by shaking in excess D-PBS overnight.

TABLE 1
C-HA scaffold compositions evaluated.

| Sample Name | Chitosan (%) | HA (%) | AA (%) |
|---|---|---|---|
| 2-0.5-0.5 | 2 | 0.5 | 0.5 |
| 2-0.5-2.5 | 2 | .5 | 2.5 |
| 4-1-1 | 4 | 1 | 1 |
| 4-4-5 | 4 | 1 | 5 |
| 4-1-10 | 4 | 1 | 10 |
| 4-2-1 | 4 | 2 | 1 |
| 4-2-5 | 4 | 2 | 5 |

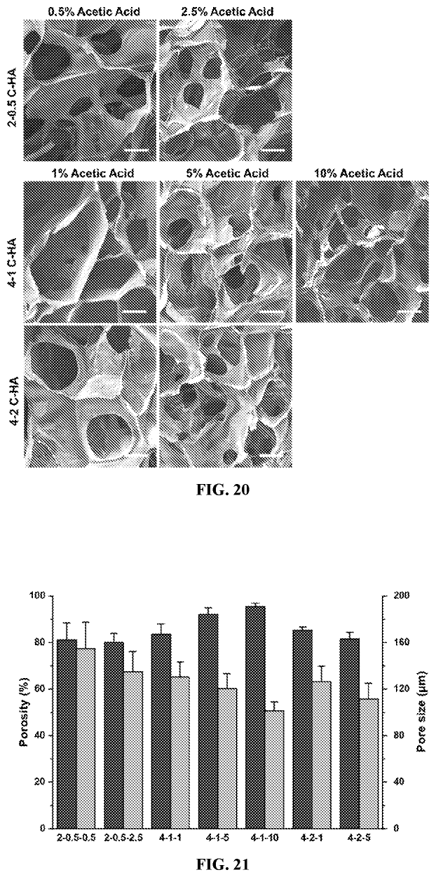

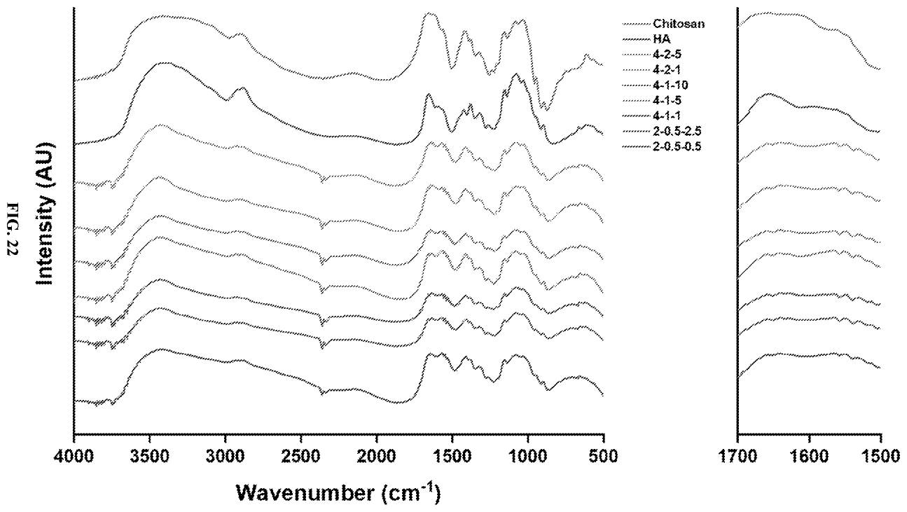

The appearance of representative C-HA scaffolds in dry and wet conditions is presented in FIG. 19. The C-HA scaffold pore morphologies demonstrated changes associated with the increasing amount of acetic acid added into the C-HA scaffolds with fixed chitosan to HA ratio (FIG. 20). The pore size decreased and smaller pores formed within the walls of the larger pores with increasing acetic acid concentrations, creating highly interconnected structures. The pore size for all C-HA scaffold compositions ranged from 100 to 160 μm and all compositions had 80% porosity or greater (FIG. 21). At a fixed chitosan-HA ratio, the pore size decreased with increasing acetic acid concentration, confirming the observations of pore morphologies in the SEM images. For example, the pore sizes of the 4C-1HA compositions decreased from 130 μm to 101 μm with increasing acetic acid concentration. The higher acetic acid concentrations also led to increased scaffold porosity, with the porosity of the 4C-1HA scaffolds increasing from 80% to 95%. However, the other C-HA compositions did not demonstrate increasing porosity with increasing acetic acid concentration and porosity remained at approximately 80%. Thus, the acetic acid concentration had a significant impact on pore structure of C-HA scaffolds and yielded smaller pores providing a more interconnected 3D network. Compared with acetic acid, differences in chitosan and HA concentrations did not affect the scaffold microstructure to the same degree as acetic acid.

FTIR analysis demonstrated that all C-HA compositions formed polyelectrolyte complexes (PECs) (FIG. 22). The C-HA compositions all showed broadening of the peaks from the chitosan and HA spectra, particularly in the 1500-1700 cm⁻¹ range, indicating PEC formation.

The viscosity analysis demonstrated increased C-HA solution viscosity with chitosan concentration (FIG. 23). There was no effect observed with increasing acetic acid concentration.

The C-HA scaffold stiffness was evaluated in dry and wet conditions. The acetic acid concentration affected the scaffold stiffness in dry conditions, with increasing acetic acid concentration resulting in softer scaffolds. The same effect was not as prevalent in wet conditions, with chitosan concentration appearing to influence the scaffold stiffness. Increased C-HA concentrations promoted increased mechanical properties in both dry and wet conditions.

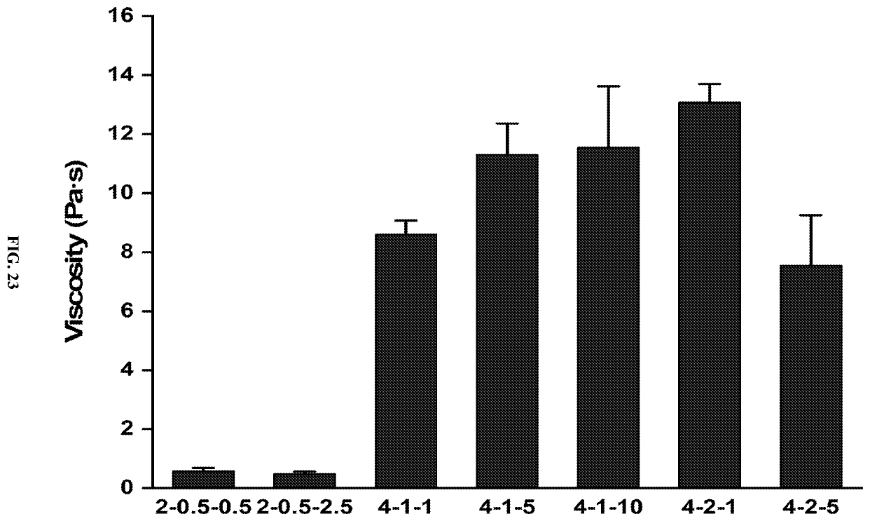

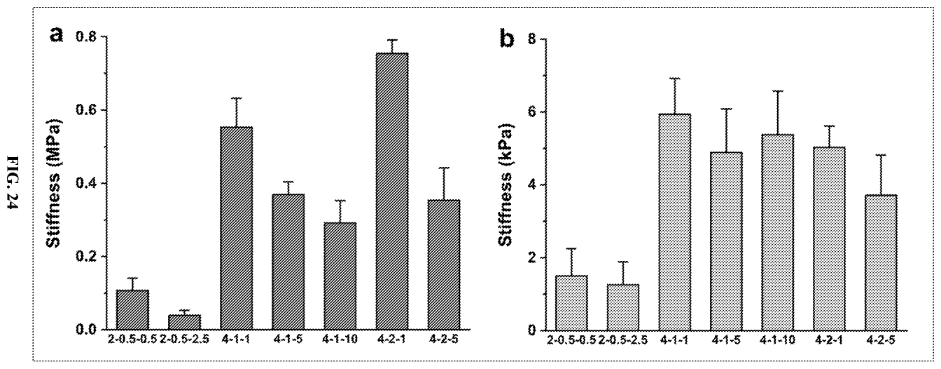

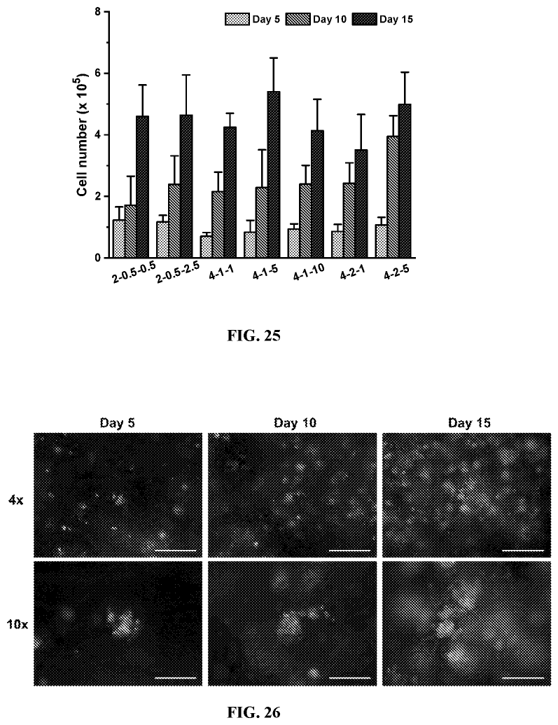

The seven C-HA scaffold compositions were cultured with MDA-MB-231-GFP cells to assess the influence of C-HA scaffold properties on cell response. The cell number in the C-HA scaffolds was analyzed with alamarBlue assay at 5, 10, and 15 day time points (FIG. 25). The cell number increased in all C-HA scaffolds during the 15 day culture.

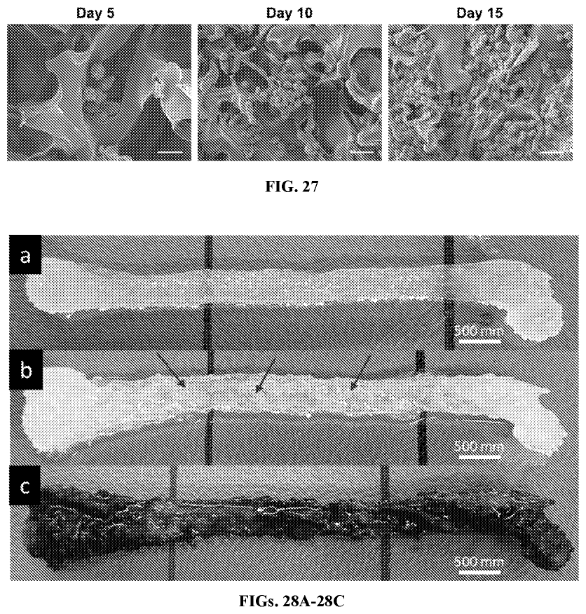

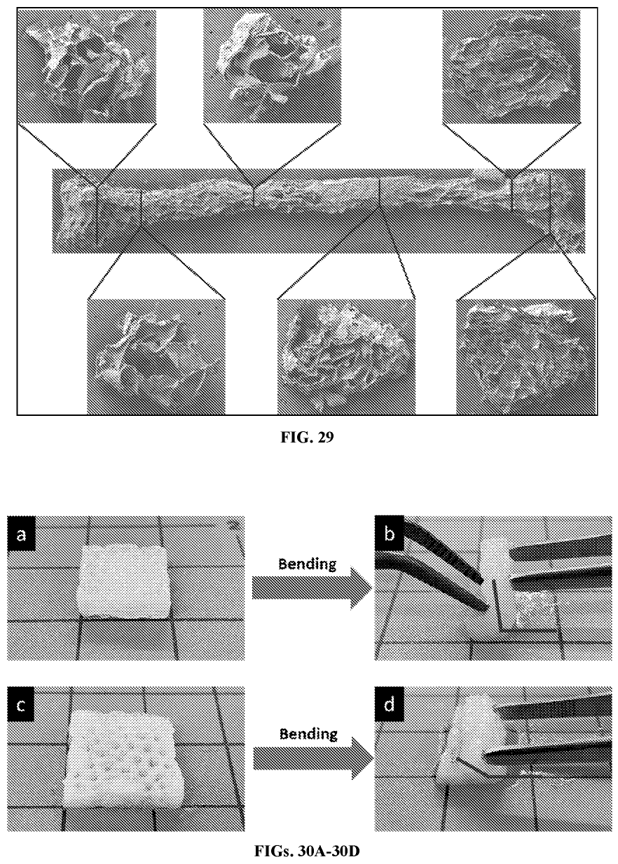

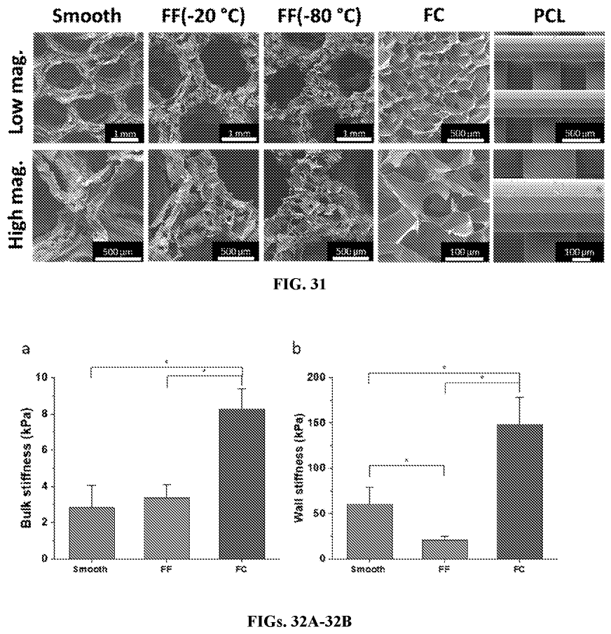

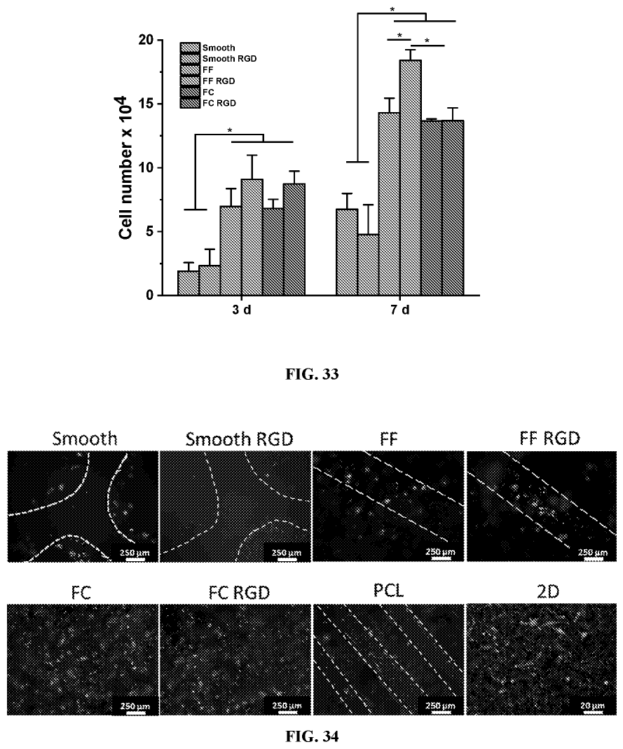

Cell morphology on C-HA scaffolds was observed using fluorescent imaging. The 231 cells aggregated and formed grape-like structures inside the interconnected pores in C-HA scaffolds as shown in FIGS. 26 and 27. The cells maintained a rounded morphology during culture and formed cell clusters as they grew on the C-HA scaffolds. The number of cell clusters increased during culture, occupying more of the pores in C-HA scaffolds and the clusters increased in size.

### Example 4: 3D Printing

The gelatin slurry support bath was prepared by adapting the published method (Hinton, T J, et al. Three-dimensional printing of complex biological structures by freeform reversible embedding of suspended hydrogels, Science Advances 1(9) (2015) e1500758). Briefly, 10 g of gelatin (Type A, Sigma-Aldrich) and 0.4 g of CaCl₂ (Sigma-Aldrich) were added into 250 ml of deionized (DI) water at 40-45° C. in a 500 ml glass mason jar (Ball Inc.) and stirred until fully dissolved. The solution was stored overnight in a 4° C. refrigerator to gel. Next, 250 ml of 0.16 w/v % CaCl₂ solution at 4° C. was added to the jar and the solution was blended on an Oster™ Heritage Blend™ 400 Blender at pulse speed for 90 s. The blended gelatin slurry was transferred into 50 ml conical tubes and diluted with cold CaCl₂ solution in a 3:2 ratio. The mixture was vortexed to wash out the soluble gelatin. After this process, the mixture was centrifuged at 3700 g at 4° C. for 2 min, causing gelatin particles to settle out of the suspension. After centrifuging, the supernatant containing a white foamy top layer of the soluble gelatin was aspirated and replaced with fresh CaCl₂ solution. The mixture was vortexed and centrifuged again to further wash out the soluble gelatin. This process was conducted several times until there was no foam visible on top of the supernatant. At this point, gelatin bath can be stored with cold CaCl₂ solution at 4° C. in suspension. For printing, the suspension was centrifuged at 225 g for 5 min to compact the gelatin slurry. The slurry was then poured into Petri dishes for printing. The Petri dish was covered with Kimwipes to remove any excess fluid. After replacing several Kimwipes the gelatin bath was ready for printing.

The inks used comprised a chitosan ink and an alginate ink. The chitosan solution was prepared at 3 w/v % chitosan (practical grade, >75% deacetylated, MW=190,000−375,000, Sigma) in 1% acetic acid. The alginate solution contained 4 w/v % sodium alginate (FMC BioPolymer) and 0.4 w/v % hyaluronic acid (hyaluronic acid sodium salt, from Streptococcus equi, Sigma) in DI water. Both solutions were mixed in a Thinky mixer (ARM-300, Thinky USA, Laguna Hills, Calif.) 2000 rpm for 3 min twice. The solutions were aged overnight at room temperature. The alginate ink was dyed with food coloring for visualization during printing.

A 3D cuboid model with dimensions of 10 mm×10 mm×4 mm was created using SolidWorks (Dassault Systémes) and opened in MeshLab (http://meshlab.sourceforge.net/) for export in the .stl file format. The digital model was loaded into Reptier-host to slice the model into layers for printing with the Slic3r plugin. The slicing pattern for the cuboid was defined to be a solid honeycomb with 40% infill density and 0.5 mm layer height, creating the design for printing. After slicing, the G code for the corresponding .stl files was exported from Repetier-host and loaded into the operating interface of the 3D printer (Biobots 1). The samples were 3D printed with the FRESH printing process using a Biobots 1 3D printer (Biobots Inc.). The ink was drawn into a 10 ml Luer-Lok syringe (BD) and printed with a 27 gauge (0.21 mm inner diameter) 0.5 inch flat tipped stainless steel needle (Jensen Global, Inc.). The syringe was loaded into the 3D printer and z-axis calibration was performed prior to printing. A 35 mm diameter Petri dish filled with the gelatin slurry support bath was placed on the printing platform. The samples were printed at room temperature (22±1° C.) with printing taking between 7 to 20 min depending on the design. After printing, the samples were frozen in a −20° C. freezer overnight (>12 h). The frozen samples were lyophilized with a freeze drier (Virtis Freezemobile) for 24 h. After drying, the printed samples were crosslinked or neutralized and recovered from the gelatin bath. The alginate prints were ionically crosslinked and recovered by immersing the prints in 0.2 M CaCl₂) solution at 37° C. on a stir plate to melt the gelatin and stabilize the structure. The chitosan prints were neutralized and recovered by immersing the prints in 1 M sodium hydroxide solution at 37° C. on a stir plate to melt the gelatin and stabilize the structure. Smooth 3D printed samples were prepared following the same method, except without freezing or lyophilization. Control samples were prepared by freeze casting, where the chitosan and alginate inks were cast in 24-well plate molds, frozen at −20° C., and lyophilized to produce 3D porous scaffolds.

TABLE 2

| | | | | | Printing parameters | | | Dimensions | Dimensions |
|---|---|---|---|---|---|---|---|---|---|
| Print | | Gelatin | 3D model | Layer | Needle | Pressure/ | Printing | before | after |
| method | Material | phase | dimensions/mm | height/mm | gauge | psi | [symbol] | recovering/mm | recovering/mm |
| By hand | 4% Alginate | No gelatin, | [symbol] | N/A | [symbol] | N/A | N/A | [symbol] | N/A |
| | low grade | blended by | | | | | | | |
| | | spatula | | | | | | | |
| By hand | 4% [symbol] | No gelatin, | [symbol] | N/A | [symbol] | N/A | N/A | [symbol] | N/A |
| | | blended by | | | | | | | |
| | | spatula | | | | | | | |
| By hand | 4% CA | No gelatin, | [symbol] | N/A | [symbol] | N/A | N/A | [symbol] | N/A |
| | | blended by | | | | | | | |
| | | spatula | | | | | | | |
| Biobots 1 | 2% Alginate | No gelatin, | Cylinder | N/A | 30 | 40 | 2 | N/A | N/A |
| | (high grade) | blended by | N/A | | | | | | |
| | | household | | | | | | | |
| | | blender | | | | | | | |
| Biobots 1 | 2% Alginate | [symbol] | [symbol] | 0.5 | 30 | 10 | 4 | [symbol] | N/A |
| | (high grade) | | | | | | | | |
| Biobots 1 | 2% Alginate | [symbol] | [symbol] | 0.5 | 30 | 10 | 4 | [symbol] | N/A |
| | (high grade) | | | | | | | | |
| Biobots 1 | 2% Alginate | [symbol] | [symbol] | 0.5 | 25 | 5 | 4 | [symbol] | N/A |
| | (high grade) | | | | | | | | |
| Biobots 1 | 4% Alginate | [symbol] | [symbol] | 0.5 | 25 | [symbol] | 4 | [symbol] | N/A |
| | low grade | | | | | | | | |
| Biobots 1 | 4% Alginate | [symbol] | [symbol] | 0.5 | [symbol] | [symbol] | 4 | [symbol] | N/A |
| | low grade | | | | | | | | |
| Biobots 1 | 4% Alginate | [symbol] | [symbol] | 0.5 | [symbol] | [symbol] | 4 | [symbol] | [symbol] |
| | low grade | | | | | | | | |

| | Further | | Swelling | | Freezing method | | Crosslinking | | |
|---|---|---|---|---|---|---|---|---|---|
| | crosslinking | | with [symbol] | Dimensions | | Within | with 0.2M [symbol] | [symbol] | Dimensions |
| Print | with 0.2M | | PBS before | after | Temperature [symbol] | gelatin | after freeze- | after freeze- | after freeze- |
| method | [symbol] | [symbol] | freezing | swelling/mm | ° C. | bath | drying | drying | drying/mm |
| By hand | No | No | No | N/A | −20 | Yes | Yes | No | N/A |
| By hand | No | No | No | N/A | −20 | Yes | No | Yes | N/A |
| By hand | No | No | No | N/A | −20 | Yes | Yes | No | N/A |
| Biobots 1 | No | No | No | N/A | −20 | Yes | Yes | No | N/A |
| Biobots 1 | No | No | No | N/A | 20 | Yes | Yes | No | [symbol] |
| Biobots 1 | No | No | No | N/A | [symbol] | Yes | Yes | No | [symbol] |
| Biobots 1 | No | No | No | N/A | 20 | Yes | Yes | No | [symbol] |
| Biobots 1 | No | No | No | N/A | −20 | Yes | Yes | No | [symbol] |
| Biobots 1 | No | No | No | N/A | −80 | Yes | Yes | No | [symbol] |
| Biobots 1 | Yes | No | Yes [symbol] | [symbol] | −20 | No | No | No | [symbol] |

[symbol]

indicates data missing or illegible when filed

TABLE 3
Dimension change of scaffolds after recovery and the
porosity of 3D printed and freeze-casted scaffolds

| Sample | Shrinkage (%) | Porosity (%) |
|---|---|---|
| Alginate smooth | 64.46 ± 17.54 | |
| Alginate frozen at −20° C. | 45.19 ± 8.78 | |
| Alginate frozen at −80° C. | 50.06 ± 5.83 | |
| Alginate freeze-casted at −20° C. | X | 84.87 ± 2.07 |
| Chitosan smooth | 38.65 ± 2.90 | |
| Chitosan frozen at −20° C. | 53.47 ± 3.02 | |
| Chitosan frozen at −80° C. | 73.79 ± 5.86 | |
| Chitosan freeze-casted at −20° C. | X | 87.89 ± 2.42 |

The materials and methods of the appended claims are not limited in scope by the specific materials and methods described herein, which are intended as illustrations of a few aspects of the claims and any materials and methods that are functionally equivalent are within the scope of this disclosure. Various modifications of the materials and methods in addition to those shown and described herein are intended to fall within the scope of the appended claims. Further, while only certain representative materials, methods, and aspects of these materials and methods are specifically described, other materials and methods and combinations of various features of the materials and methods are intended to fall within the scope of the appended claims, even if not specifically recited. Thus a combination of steps, elements, components, or constituents can be explicitly mentioned herein; however, all other combinations of steps, elements, components, and constituents are included, even though not explicitly stated.

1. An aqueous cell culture scaffold solution, comprising: chitosan and alginate, chitosan and hyaluronic acid, or chitosan and chondroitin sulfate, wherein the solution comprises greater than 4 wt % of chitosan.

2. (canceled)

3. (canceled)

4. (canceled)

5. The solution of claim 1, wherein the solution comprises greater than 5 wt % chitosan.

6. The solution of claim 1, wherein the ratio of chitosan to alginate, hyaluronic acid, or chondroitin sulfate is 1:1, 2:1, or 4:1.

7. A dried cell culture scaffold composition, comprising: a dried solution of chitosan and alginate, chitosan and hyaluronic acid, or chitosan and chondroitin sulfate, wherein the solution comprises greater than 4 wt % of chitosan.

8. The dried composition of claim 7, wherein the stiffness of the composition is greater than 2 MPa.

9. (canceled)

10. (canceled)

11. (canceled)

12. The dried composition of claim 7, wherein the solution comprises greater than 5 wt % chitosan.

13. The dried composition of claim 7, wherein the ratio of chitosan to alginate, hyaluronic acid, or chondroitin sulfate is 1:1, 2:1, or 4:1.

14. The dried composition of claim 7, wherein the scaffold is a 3D scaffold.

15. The dried composition of claim 7, wherein the composition is sterilized.

16. A cell culture, comprising: a cancer cell, a growth medium, and a scaffold, wherein the scaffold comprises chitosan and alginate, chitosan and hyaluronic acid, or chitosan and chondroitin sulfate, and wherein the scaffold is prepared from the aqueous solution of claim 1.

17. The cell culture of claim 16, wherein the cancer cells are from a patient derived xenograft.

18. The cell culture of claim 16, wherein the cancer cells are resistant to Docetaxel, Xtandi, Zytiga, and/or cabazitaxel.

19. (canceled)

20. The cell culture of claim 16, further comprising cells of one or more non-cancer cells types.

21. A method of making a cell culture scaffold, comprising: combining an aqueous solution of chitosan and an aqueous solution of alginate, hyaluronic acid, or chondroitin sulfate to provide an aqueous scaffold solution of claim 1; removing the water to thereby provide a dried cell culture scaffold.

22. The method of claim 21, further comprising sterilizing the dried scaffold composition.

23. The method of claim 21, wherein the water is removed by freeze drying.

24. The method of claim 21, wherein the dried cell culture scaffold is crosslinked by adding CaCl₂.

25. A method of assaying a patient's response to a cancer therapeutic: comprising, providing a cell culture scaffold comprising chitosan and alginate, chitosan and hyaluronic acid, or chitosan and chondroitin sulfate, wherein the scaffold is formed by the method of claim 21; contacting the cell culture scaffold with cancer cells derived from the patient and culture medium; culturing the cells, and contacting the cell culture scaffold with a putative therapeutic.

26. A method of characterizing a patient's cancer cells: comprising, providing a cell culture scaffold comprising chitosan and alginate, chitosan and hyaluronic acid, or chitosan and chondroitin sulfate, wherein the scaffold is formed by the method of claim 21; contacting the cell culture scaffold with cancer cells derived from the patient and culture medium; culturing the cancer cells; and removing the cancer cells from the cell culture scaffold, and characterizing the phenotype or metastatic potential of the cancer cells.

27. A method of preparing a 3D cell culture scaffold, comprising: extruding a scaffold composition comprising chitosan and alginate, hyaluronic acid, or chondroitin sulfate, wherein the scaffold comprises greater than 4 wt % of the scaffold composition onto a substrate material, wherein the location of the scaffold composition extruded onto the substrate material is controlled by an X-Y-Z movement system, and wherein the substrate material comprises gelatin beads; freezing the scaffold composition and gelatin beads; removing ice and water from the frozen scaffold composition and gelatin beads; crosslinking or neutralizing the scaffold composition; and removing the gelatin from the crosslinked or neutralized scaffold composition, thereby providing a 3D cell culture scaffold.
